(19) United States
(12) Patent Application Publication
(10) Pub. No.: US 2023/0086099 A1
(43) Pub. Date: Mar. 23, 2023

(54) COMBINATION THERAPY FOR TREATMENT OF CANCER AND CANCER METASTASIS

(71) Applicant: ONA Therapeutics, S.L., Barcelona (ES)

(72) Inventors: Salvador AZNAR BENITAH, Barcelona (ES); Pier GIORGIO AMENDOLA, Barcelona (ES); Valerie VANHOOREN, Barcelona (ES); Mercè DE FRIAS SÁNCHEZ, Barcelona (ES); Beatriz MORANCHO ARMISEN, Barcelona (ES)

(73) Assignee: ONA Therapeutics, S.L., Barcelona (ES)

(21) Appl. No.: 17/795,814

(22) PCT Filed: Jan. 29, 2021
(86) PCT No.: PCT/IB2021/050747

§ 371 Date: Jul. 27, 2022

Related U.S. Application Data
(60) Provisional application No. 62/967,875, filed on Jan. 30, 2020.

(30) Foreign Application Priority Data
Jan. 30, 2020 (EP) 20382054.3

(51) Int. Cl. C07K 16/28; A61P 35/00

(52) U.S. Cl. CPC C07K 16/2896; A61P 35/00; A61K 2039/507

(57) ABSTRACT

Targeting cancer through a fatty acid receptor. The invention provides the use of blockers or inhibitors of CD36 activity or expression for the treatment of cancer in combination with a second therapy.

### TECHNICAL FIELD

The disclosure relates to the treatment of cancer, particularly cancer metastases, and the control of said disease. More specifically, the disclosure relates to the use of antibodies and other inhibitors of CD36 activity or expression for the treatment of cancer, particularly cancer metastases, in combination with a second therapy such as chemotherapy or immunotherapy.

### BACKGROUND

CD36 (HGNC: 1663, EntrezGene: 948, Ensembl: ENSG00000135218, OMIM: 173510, UniProtKB: P16671) is a receptor protein with several different known functions, as it is indicated by the different alternative names that it receives: it is known, among others, as cluster determinant 36, thrombospondin receptor, collagen type I receptor, leukocyte differentiation antigen CD36, platelet glycoprotein 4 or fatty acid translocase. The Entrez Gene and UniProt/SwissProt Summaries for CD36 gene, as recapitulated by GeneCards (http://www.genecards.org/cgi-bin/carddisp.pl?gene=CD36) describe the protein as the fourth major glycoprotein of the platelet surface that serves as a receptor for thrombospondin in platelets and various cell lines. Since thrombospondins are widely distributed proteins involved in a variety of adhesive processes, this protein may have important functions as a cell adhesion molecule. It binds to collagen and thrombospondin, mediating the antiangiogenic effect of the latter, as well as to anionic phospholipids and oxidized LDL. It directly mediates cytoadherence of Plasmodium falciparum parasitized erythrocytes and it binds long chain fatty acids and may function in the transport and/or as a regulator of fatty acid transport. It is a co-receptor for TLR4-TLR6 heterodimer that promotes inflammation in monocytes/macrophages. Upon ligand binding, such as oxLDL or amyloid-beta 42, rapidly induces the formation of a heterodimer of TLR4 and TLR6, which is internalized and triggers an inflammatory response, leading to NF-kappa-B-dependent production of CXCL1, CXCL2 and CCL9 cytokines, via MYD88 signalling pathway, and CCL5 cytokine, via TICAM1 signalling pathway, as well as IL1b secretion. CD36 is also at the top of the signalling cascade that uptakes lipids from the extracellular environment and triggers their beta-oxidation to obtain energy in the form of ATP (Coburn et al., 2000; Ibrahimi et al., 1999; Pepino et al., 2014).

CD36 has been previously related to cancer, but its implication for therapy and mechanism of action were not clear.

WO 03/032813 discloses assays where it is shown that CD36 is one of the genes upregulated in renal cell carcinoma. Although no assays are presented for other types of cancer, CD36 is presented in said application as a useful target for the diagnosis and/or treatment, and even prevention, of certain cancers, being also considered as a predictor of the prognosis of the tumour treatment. Squamous cell carcinoma (SCC) is mentioned as one of the possible cancer types where the treatment with CD36 antibodies, or antagonists such as antisense RNA, can be of use, but without providing any evidence of changes of CD36 expression in SCC or, particularly, of the efficacy of CD36 antibodies or other antagonists for preventing or treating either primary tumours or metastases. Spontaneous animal tumours are proposed for testing the efficacy of antibodies specifically binding the proteins that are overexpressed in renal cell carcinoma according to the assays shown in WO 03/032813, and, given that it is a highly invasive and malignant tumour, feline oral SCC is proposed as a suitable model. However, again, such proposal is done without providing examples of the actual utility of said approach and moreover, without showing any evidence that any of the genes overexpressed in renal cell carcinoma are also overexpressed in feline oral SCC and, particularly, not showing either any data about changes (increase or decrease) in the level of expression of CD36 in feline oral SCC or any evidence about a possible involvement of CD36 in the initiation, development or spread of metastasis in such type of cancer. Moreover, it is commented that feline oral SCC exhibits low incidence of metastasis, but also mentioning that this might be due to the short survival times of cats with this tumour.

For breast cancer, some authors (DeFillippis et al., 2012) have reported that CD36 repression activates a multicellular stromal program shared by high mammographic density and tumour tissues, so that the decrease/repression of CD36 makes tumours more aggressive. They show that increased expression of CD36 can restore stromal phenotypes associated with low risk tissues.

The available data indicate that the role of CD36 in different kinds of cancer, if any, might be different and opposed depending on the particular kind of cancer considered and, even, on the particular stage of said cancer. Some authors (Balaban et al., 2015) had suggested that the multifunctional character of CD36 might be associated with the different role of changes in CD36 expression depending on the cancer type. They mention that low CD36 gene expression correlates with a higher metastasis grade in colon and ovarian cancers and with low recurrence-free survival but, conversely, CD36 mRNA expression in breast cancer is inversely correlated with the metastatic potential of five breast cancer cell lines, where its expression is relatively higher in less aggressive cell lines and almost absent in highly aggressive lines (ZR-75 and MDA-MB-231). This inconsistency between cancer types may be explained by the multifunctionality of CD36. While it functions as a fatty acid transporter, CD36 is also involved in collagen adhesion and, therefore, lower expression of CD36 may lead to reduced cell adhesion, providing cancer cells with a higher metastatic potential. They suggest that the rate of fatty acid uptake mediated by CD36 in each particular case might also have an important implication in the effect on cancer progression, and that it might be influenced by an obese microenvironment.

Other groups have suggested a role of oxidized lipids in the metabolism and functionality of cancer cells. They are broadly regarded as compounds with a cytotoxic effect (Alghazeer et al., 2008), so that an excessive uptake of oxidized lipids may lead to a reduced viability of cancer cells and even to apoptosis.

The involvement of lipid uptake and metabolism in cancer progression has been discussed by other research groups. It is generally considered that cancer cells, that are usually cells with a high rate of division, have an altered energetic metabolism, so that glucose and lipids are metabolized differently than in normal cells. The specific modifications in lipid metabolism in cancer cells have not been clearly identified, and it has not been studied in developed metastasis.

With regard to metastasis, it has been previously shown that inhibition of CD36 (both by antibodies neutralizing its activity or by shRNAs) has a dramatic effect regarding metastasis initiation and progression, decreasing metastatic penetrance and growth of all cell lines and patient-derived tumours tested. See, U.S. Publ. No. 2019-0106503, which is incorporated herein by reference in its entirety.

Programmed Cell Death 1 (PD-1) is a cell surface signaling receptor that plays a critical role in the regulation of T cell activation and tolerance (Keir M. E., et al., Annu. Rev. Immunol. 2008; 26:677-704). It is a type I transmembrane protein and together with BTLA, CTLA-4, ICOS and CD28, comprise the CD28 family of T cell co-stimulatory receptors. PD-1 is primarily expressed on activated T cells, B cells, and myeloid cells (Dong H., et al., Nat. Med. 1999; 5:1365-1369). It is also expressed on natural killer (NK) cells (Terme M., et al., Cancer Res. 2011; 71:5393-5399). Binding of PD-1 by its ligands, PD-L1 and PD-L2, results in phosphorylation of the tyrosine residue in the proximal intracellular immune receptor tyrosine inhibitory domain, followed by recruitment of the phosphatase SHP-2, eventually resulting in down-regulation of T cell activation. One important role of PD-1 is to limit the activity of T cells in peripheral tissues at the time of an inflammatory response to infection, thus limiting the development of autoimmunity (Pardoll D. M., Nat. Rev. Cancer 2012; 12:252-264). Evidence of this negative regulatory role comes from the finding that PD-1-deficient mice develop lupus-like autoimmune diseases including arthritis and nephritis, along with cardiomyopathy (Nishimura H., et al., Immunity, 1999; 11:141-151; and Nishimura H., et al., Science, 2001; 291:319-322). In the tumor setting, the consequence is the development of immune resistance within the tumor microenvironment. PD-1 is highly expressed on tumor-infiltrating lymphocytes, and its ligands are up-regulated on the cell surface of many different tumors (Dong H., et al., Nat. Med. 2002; 8:793-800). Multiple murine cancer models have demonstrated that binding of ligand to PD-1 results in immune evasion. In addition, blockade of this interaction results in anti-tumor activity (Topalian S. L., et al. NEJM 2012; 366(26):2443-2454; Hamid O., et al., NEJM 2013; 369:134-144). Moreover, it has been shown that inhibition of the PD-1/PD-L1 interaction mediates potent antitumor activity in preclinical models (U.S. Pat. Nos. 8,008,449 and 7,943,743).

### SUMMARY

In some embodiments, the disclosure is directed to a method of treating cancer in a subject comprising administering to the subject in need thereof a therapeutically effective amount of: a CD36 inhibitor; and a second therapy. In some embodiments, the cancer is selected from the group consisting of: oral squamous cell carcinoma (OSCC), head and neck cancer, esophageal cancer, gastric cancer, ovarian cancer, cervical cancer, lung cancer, breast cancer, colon cancer, renal cancer, prostate cancer, sarcoma, melanoma, leukemia, and lymphoma. In some embodiments, the cancer is selected from the group consisting of: oral squamous cell carcinoma, ovarian cancer, colon cancer, lung cancer, and melanoma. In certain embodiments, the cancer is metastatic cancer. In some embodiments, the cancer comprises one or more metastatic tumors present in one or more of the liver, lung, spleen, kidney, cervical lymph nodes, or peritoneal wall. In certain embodiments, the cancer is a primary tumor. In some embodiments, the subject is a human.

In some embodiments, the CD36 inhibitor is an antibody, a single chain antibody, or a scFv, Fab or F(ab′)2 fragment. In certain embodiments, the CD36 inhibitor is an antibody. In some embodiments, the CD36 inhibitor is a humanized antibody. In some embodiments, the CD36 inhibitor is a human antibody. In some embodiments, the CD36 inhibitor is a shRNA or an iRNA, a siRNA, or an antisense RNA or DNA.

In certain embodiments, the second therapy is an immunotherapy. In some embodiments, the immunotherapy is a PD-1 inhibitor. In some embodiments, the PD-1 inhibitor is an anti-PD-1 antibody. In certain embodiments, the anti-PD-1 antibody is pembrolizumab (KEYTRUDA; MK-3475), pidilizumab (CT-011), or nivolumab (OPDIVO; BMS-936558). In some embodiments, the immunotherapy is a PD-L1 inhibitor. In some embodiments, the PD-L1 inhibitor is an anti-PD-L1 antibody. In certain embodiments, the anti-PD-L1 antibody is atezolizumab (Tecentriq or RG7446), durvalumab (Imfinzi or MEDI4736), avelumab (Bavencio) or BMS-936559. In some embodiments, the immunotherapy is a CTLA-4 inhibitor. In certain embodiments, the CTLA-4 inhibitor is an anti-CTLA-4 antibody. In some embodiments, the anti-CTLA-4 antibody is ipilimumab or an antigen-binding fragment thereof.

In some embodiments, the second therapy is one or more chemotherapeutic agents. In some embodiments, the chemotherapeutic agent is cisplatin.

In certain embodiments, metastasis is reduced or inhibited in the subject. In some embodiments, the number of metastases is reduced. In some embodiments, the growth of one or more tumors is inhibited. In some embodiments, the growth of one or more metastatic tumors is inhibited. In some embodiments, the treatment reduces the size of metastatic tumors, as measured by an in vivo imaging system (IVIS) or by H&E staining. In some embodiments, the growth of one or more metastatic tumors is inhibited In some embodiments, the treatment increases the amount of necrosis in one or more tumors. In some embodiments, the treatment increases the amount of fibrosis in one or more tumors.

In some embodiments, the two therapies are administered sequentially. In some embodiments, the two therapies are administered simultaneously.

### BRIEF DESCRIPTION OF THE FIGURES

FIGS. 1A-1E show that anti-CD36 Ab treatment enhanced anti-tumor activity on the primary tumor when combined with cisplatin to treat oral cancer.

FIG. 2 shows that combined anti-CD36 Ab and cisplatin treatment reduces both the size and number of lung metastases. All analysis were done based on H&E staining of the lungs and scored blindly by a mouse pathologist, with representative pictures shown.

FIGS. 3A and 3B show anti-CD36 Ab treatment has a different method of action and complementary anti-tumor activity compared to cisplatin. When anti-CD36 was combined with cisplatin to treat lung metastases from oral cancer, anti-CD36 Ab reduced the number and size of metastases while cisplatin reduced the size of metastases.

FIGS. 4A-4E show anti-CD36 antibody is effective in treating lymph node metastases as a monotherapy, and that anti-CD36 antibody has a synergistic effect with cisplatin in combination therapy, in a mouse model using the aggressive FaDu cell line (oral cancer cell line).

FIGS. 5A-5E and FIGS. 6A-6B show lymph node metastasis in cisplatin treated mice, CD36 Ab treated mice, and cisplatin+CD36 Ab treated mice, and show that the ONA-0 anti-CD36 antibody is effective as a monotherapy or as part of a combination therapy with cisplatin.

FIG. 7A is a schematic showing an experimental overview of a study of the effects of the ONA-0 anti-CD36 antibody in combination with cisplatin in a mouse model of ovarian cancer using OVCAR-3 cells. FIG. 7B details the study groups tested in that study, particularly the therapeutics and doses given to each group. The results of the study described in FIGS. 7A and 7B are depicted in FIGS. 8A-8B and 9A-9C.

FIGS. 8A and 8B depict the quantification of the number and size of metastases in the OVCAR-3 mouse model of ovarian cancer in cisplatin-treated mice and mice treated with cisplatin and ONA-0. FIG. 8A shows the percentage of mice with metastasis per group based on macroscopic quantification of metastases in the peritoneal wall and liver, respectively. FIG. 8B shows the microscopic quantification of the number and size of metastases in the liver. Collectively, FIGS. 8A and 8B show that treating with ONA-0 decreases the size and number of metastases in the OVCAR-3 mouse model of ovarian cancer.

FIG. 9A shows images of primary tumors excised from mice tested in the model described in FIGS. 7A-7B, with tumors from cisplatin-injected mice on the top row and tumors from mice injected with cisplatin and ONA-0 on the bottom row. FIG. 9B presents the quantification of the weight of these primary tumors, and shows that treatment with ONA-0 in combination with cisplatin resulted in a relative decrease in the weight of the primary tumors. FIG. 9C shows the results of histological analysis of the OVCAR-3 primary tumors for percent necrosis and fibrosis/collagen, respectively. FIG. 9C also shows that treatment with cisplatin and ONA-0 results in increased necrosis and fibrosis in the analyzed tumors.

FIG. 10A is a schematic showing an experimental overview of a study of the effects of the 1G04 anti-CD36 antibody in combination with anti-PD-1 in a mouse model of metastatic colon cancer using MC-38 cells. FIG. 10B details the study groups tested in that study, particularly the therapeutics and doses given to each group. The results of the study described in FIGS. 10A and 10B are depicted in FIGS. 11A-11B.

FIG. 11A shows the quantification in vivo of the luciferase luminescence from within the MC-38 cells during the course of treatment. FIG. 11B shows that 1G04 treatment in combination with anti-PD-1 reduces the number of macrometastasis in the liver and the liver weight in the MC-38 mouse model of colon cancer.

### DETAILED DESCRIPTION

The present disclosure related to methods of treating (e.g., reducing and/or inhibiting) cancer, particularly cancer metastases, by administering a CD36 inhibitor and a second therapy. In some embodiments, the CD36 inhibitor is an anti-CD36 antibody. In particular embodiments, the second therapy is an immunotherapy. In some embodiments, the second therapy is a chemotherapy or a chemotherapeutic agent. In some embodiments, the immunotherapy is an anti-PD-1 antibody. In some embodiments, the second therapy is a chemotherapeutic agent. In some embodiments, the chemotherapy or chemotherapeutic agent is cisplatin.

### Definitions of General Terms and Expressions

“And/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

As used herein, “antibody”, “antibody molecule”, or “antibodies” describes an immunoglobulin whether naturally, or partly, or wholly synthetically produced. The term also covers any polypeptide or protein comprising an antibody antigen-binding site. It must be understood here that the invention does not relate to the antibodies in natural form, that is to say they are not in their natural environment but that they have been able to be isolated or obtained by purification from natural sources, or else obtained by genetic recombination, or by chemical synthesis, and that they can then contain unnatural amino acids. Antibody fragments that comprise an antibody antigen-binding site include, but are not limited to, molecules such as Fab, Fab′, F(ab′)2, Fab′-SH, scFv, Fv, dAb and Fd. Various other antibody molecules including one or more antibody antigen-binding sites have been engineered, including for example Fab2, Fab3, diabodies, triabodies, tetrabodies, camelbodies, nanobodies and minibodies. Antibody molecules and methods for their construction and use are described in Hollinger & Hudson (2005) Nature Biot. 23(9): 1126-1136.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

“Administering” refers to the physical introduction of a composition comprising a therapeutic agent to a subject, using any of the various methods and delivery systems known to those skilled in the art. Preferred routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, preferably orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

### CD36 Inhibitors

As they are used herein, the terms “CD36 blocker” and “CD36 inhibitor” include any compound, or salt thereof, that reduces or abolishes the activity of its target, in this case, CD36. The term blocker is often used as a synonym for an inhibitor, and vice versa. The terms blocker and inhibitor are also used as synonyms of the term receptor antagonist. As a reduction or complete inhibition of expression also gives rise to a reduction of the activity of the non-expressed protein, the terms CD36 blocker and CD36 inhibitor, as they are used herein, also encompass those compounds that inhibit, partially or completely, the expression of the CD36 gene. Thus, the terms CD36 blocker and CD36 inhibitor encompass both those compounds that directly interfere with CD36 activity and those compounds that reduce CD36 expression. A compound that can be a CD36 blocker or CD36 inhibitor suitable for the purposes of the present invention can be a small organic molecule, that is, a molecule of a size comparable to those organic molecules generally used in pharmaceuticals, which organic molecules can be natural but that are often obtained by chemical synthesis or modification or natural molecules, and which usually exhibit a size of up to about 5000 Da, provided that such molecule is capable of blocking, reducing or inhibiting the activity and/or expression of CD36. The terms CD36 blocker and CD36 inhibitor also encompass biological molecules, fragments or analogues thereof of very different sizes, again with the provision that they are capable of blocking, reducing or inhibiting the activity and/or expression of CD36. Antibodies, for instance, which are formed by four polypeptide chains connected at some points by covalent bonds giving a single molecule and that often are capable of blocking or inhibiting the activity of CD36, are included within the group of compounds that may be a CD36 blocker or CD36 inhibitor. Other biological compounds, such as those molecules formed by a number of units of nucleotides or analogues thereof, particularly oligonucleotides or analogues thereof such as shRNAs, siRNAs or antisense RNAs or DNAs, are also encompassed within the meaning of the terms CD36 blocker and CD36 inhibitor.

In embodiments, the CD36 inhibitor or blocker is an anti-CD36 antibody, a single chain antibody, or a scFv, Fab or F(ab′)2 fragment. In some embodiments, the anti-CD36 inhibitor is an antibody. In some embodiments, the CD36 inhibitor is a humanized antibody. In some embodiments, the CD36 inhibitor is a partially human antibody. In some embodiments, the CD36 inhibitor is a human antibody (i.e., a fully human antibody). In one embodiment, the CD36 antibody is neutralizing monoclonal anti-CD36 FA6.152 (Abcam, ab17044) (see, e.g., (Kermovant-Duchemin, et al., Nat. Med. 11(12):1339-1345 (2005); Mwaikambo et al., Investigative Ophthalmology & Visual Science October 47:4356-4364 (2006)). In another embodiment, the CD36 antibody is monoclonal anti-CD36 JC63.1 (CAYMAN, CAY-10009893-500) (see, e.g., (Kermovant-Duchemin, et al., Nat. Med. 11(12):1339-1345 (2005); Mwaikambo et al., Investigative Ophthalmology & Visual Science October 47:4356-4364 (2006)). In embodiments, the CD36 antibody is 5-271 (Biolegend). In some embodiments, the CD36 antibody is ab133625, ab80080, ab221605, ab64014, ab23680, ab17044, ab252922, ab124515, ab255331, ab252923, ab255332, ab76521, ab82405, ab39022, ab213064, ab269351, or ab253250 (Abcam). In embodiments, the CD36 antibody is AF1955 (R&D systems). In embodiments, the CD36 inhibitor is any CD36 antibody known in the art. In embodiments, the CD36 antibody is any antibody disclosed in U.S. Publ. No. 2019-0106503, which is incorporated herein by reference in its entirety. In embodiments, the CD36 antibody is any antibody disclosed in U.S. Patent Application Nos. 62/986,174 or 63/117,529, which are also incorporated herein by reference in their entirety.

In embodiments, the blocker can be an inhibitor of expression of CD36. An “inhibitor of expression” refers to a natural or synthetic compound that has the effect of inhibiting or significantly reducing the expression of a gene, which gene, for the purposes of the present invention, will be the CD36 gene. One or more shRNA or siRNA can be used. Both kinds of compounds are well known possible inhibitors of gene expression. They can be also expressed from other suitable vectors, insertional or non-insertional, well known by those skilled in the art. A variety of shRNAs for human CD36 (and even for other species, such as mouse) are commercially available from different providers, such as Sigma-Aldrich, that also provides siRNAs. A siRNA (small interference RNA) is a double stranded small (20-25 nucleotides) RNA that operates within the RNA interference pathway and interferes with the expression of specific genes with complementary nucleotide sequences by degrading RNA after transcription, resulting in no translation. When siRNAs are used, they can be expressed from vectors administered to the subject, or they can be administered in compositions with suitable excipients selected depending on the intended administration route. Different shRNAs or siRNA can be designed with the aid of known algorithms and methodologies such as the one described, for instance, in the web site of the Broad Institute (http://www.broadinstitute.org/rnai/public/resources/rules). In embodiments, the shRNA or siRNA is any shRNA or siRNA disclosed in U.S. Publ. No. 2019-0106503, which is incorporated herein by reference in its entirety.

As would be obvious to those skilled in the art, antisense therapy can be administered in any method disclosed herein for that same purpose, by synthesizing a RNA or DNA molecule, usually an oligonucleotide, or an analogue thereof, whose base sequence is complementary to the gene's messenger RNA and that will bind to said messenger RNA and inactivate it, turning the gene “off” because the mRNAs molecules have to be single-stranded to be translated. When administering oligonucleotides in a composition, it is preferable to use analogues thereof, that is, oligonucleotides where the nucleotide units have some chemical modification to their structure. Such modifications are usually in the sugar moiety and/or in the phosphate bond, and include the addition of one or more non-nucleotide moieties. The interest of such modification is that they usually render the molecule more resistant to nucleases, such as: the commonly used phosphorothyoate bonds instead of the phosphate bonds; modifications at the 2′ position of the sugar moiety such as 2′-O-methyl or 2′-O-methoxyethyl modifications; modifications where the ribose exhibits a link connecting the oxygen at 2′ with the carbon at 4′, thus blocking the ribose in the conformation 3′-endo (LNAs: locked nucleic acids); the replacement of the sugar backbone by an amide-containing backbone such as an aminoethylglycine backbone, as in peptide nucleic acids (PNAs); use of PMOs (nucleic acids where the ribose moiety is replaced by a morpholine group); and other modifications well known by those skilled in the art that can be found reviewed, for instance, by Kole et al. (2012). Further modifications, such as the attachment of one or more cholesterol moieties at one or both ends of the molecules, can facilitate the entering of the molecule in the cells. The design of antisense molecules can be obvious for those skilled in the art from the sequence of CD36 mRNA molecule and reviews such as the one of Kole et al. previously mentioned.

It is preferred that the CD36 blocker or CD36 inhibitor is a compound or molecule that modulates the activity of CD36, antagonizing or blocking it. Any CD36 receptor antagonist or inverse agonist could be used. As used herein, a receptor antagonist is a receptor ligand or drug that blocks or hinders agonist-mediated responses; as agonists are the compounds that bind to a receptor and activate the receptor to produce a biological response, antagonists, by blocking the action of the agonists, also block, inhibit or diminish the activity of the receptor. An inverse agonist is a compound that binds to the same receptor as the agonist but exerts the opposite effect; inverse agonists have the ability to decrease the constitutive level of receptor activation in the absence of an agonist. The compound that blocks or inhibits CD36 activity can be an antibody, preferably a specific antibody. It is also possible to use analogues or fragments of antibodies, such as single chain antibodies, single chain variable domain fragments (scFv), F(ab′)₂ fragments (which can be obtained by pepsin digestion of an antibody molecule), or Fab fragments (which can be obtained by reducing the disulphide bridges of the F(ab′)₂ fragments. Humanized antibodies can be used when the subject is a human being.

As CD36 has several known functions, the antibody can be selected so that it inhibits all known functions of CD36, including its interaction with thrombospondin, collagens and fatty acids (as happens, for example, with the antibody FA6.152 used in assays shown U.S. Publ. No. 2019-0106503) or only specific functions, as antibody JC63.1 also used in assays disclosed U.S. Publ. No. 2019-0106503, which only blocks fatty acid and oxidised-LDL uptake.

When the subject to be treated is a human being, any known anti-CD36 antibody can be used or the antibody can be prepared for being administered to human beings. For antibodies that have been generated in a non-human immune system (as those used in the assays of the present application), such as in mice, humanization can be necessary to enable their administration to human beings, in order to avoid adverse reactions. Humanized antibodies are antibodies, usually monoclonal antibodies, initially generated in a non-human species and whose protein sequences have been modified to increase their similarity to antibody variants produced naturally in humans, so that minimal sequence derived from non-human immunoglobulins remain. Even after humanization, the amino acid sequence of humanized antibodies is partially distinct from antibodies occurring naturally in human beings. Several processes are known for those skilled in the art for antibody humanization, as it has been reviewed, for instance, by Almagro and Fransson (2008), including: humanizing through production of a mouse-human (mouse Fab spliced to human Fc) chimera, which chimera might be further humanized by selective alteration of the amino acid sequence of the Fab portion; insertion of one or more CDR segments of the “donor” (non-human antibody) by replacing the corresponding segments of a human antibody, which can be done using recombinant DNA techniques to create constructs capable of expression in mammalian cell culture, or even avoiding the use of non-human mammals by creating antibody gene libraries usually derived from human RNA isolated from peripheral blood and displayed by micro-organisms or viruses (as in phage display) or even cell free extracts (as in ribosome display), selection of the appropriate intermediate product (usually, antibody fragments such as Fab or scFv) and obtaining full antibodies for instance, again, recombinant DNA techniques. Several patent documents have been dedicated to humanization methods like, for instance U.S. Pat. No. 6,054,297, assigned to Genentech; U.S. Pat. Nos. 5,225,539 and 4,816,397 are also useful references, and are incorporated herein by reference in their entirety.

The method for obtaining monoclonal antibodies is well known for those skilled in the art. In general, antibodies against CD36 receptor can be raised according to known methods, such as those mentioned in classic laboratory manuals as “Antibodies: A Laboratory Manual, Second edition”, edited by E.A. Greenfield in 2014, by administering CD36 whole protein or a fragment or epitope thereof to a host animal which is a different from the mammal where a therapeutic effect is sought. Monoclonal antibodies in particular can be prepared and isolated by any technique that provides for the production of antibody molecules by continuous cell lines in culture, such as the hybridoma technique originally described by Kohler and Milstein (1975), the human B-cell hybridoma technique (Cote et al., 1983), or the EBV-hybridoma technique (Cole et al., 1985). Alternatively, as commented above, Fab and/or scFv expression libraries can be constructed to allow rapid identification of fragments having the desired specificity to the CD36 receptor.

For the design of antibodies with a particular specificity, it is advantageous to resource to annotated NCBI Reference Sequence (NC_000007.14, Homo sapiens annotation release: 107, which is the current release on 29 Sep. 2015) or UniProtKB P16671, in order to choose as immunogen, if wished, a particular domain or region of the antibody to be targeted or mutated before generating the antibodies.

For achieving a therapeutic effect, the compound, which is a blocker or inhibitor of activity and/or expression of CD36, will be administered preferably in therapeutically effective amounts. An “effective dose” or a “therapeutically effective amount” is an amount sufficient to exert a beneficial or desired clinical result. The precise determination of what would be considered an effective dose may be based on factors individual to each patient, including their size, age, cancer stage, and nature of the blocker (e.g. expression construct, antisense oligonucleotide, antibody or fragment thereof, etc.). Therefore, dosages can be readily ascertained by those of ordinary skill in the art from this disclosure and the knowledge in the art. Multiple doses can be also administered to the subject over a particular treatment period, for instance, daily, weekly, monthly, every two months, every three months, or every six months. In certain dose schedules, the subject receives an initial dose at a first time point that is higher than one or more subsequent or maintenance doses.

### Methods of the Disclosure

In some embodiments, the present invention provides methods of treating cancer in a subject using a combination of a CD36 inhibitor and a second therapy. In some embodiments, the cancer is oral squamous cell carcinoma, head and neck cancer, esophageal cancer, gastric cancer, ovarian cancer, cervical cancer, lung cancer, breast cancer, colon cancer, renal cancer, prostate cancer, sarcoma, melanoma, leukemia, or lymphoma. In some embodiments, the cancer is oral squamous cell carcinoma. In some embodiments, the cancer is ovarian cancer. In some embodiments, the cancer is colon cancer. In some embodiments, the cancer is lung cancer. In some embodiments, the cancer is melanoma. In a further embodiment, the cancer is any cancer disclosed herein. In one embodiment, the cancer is metastatic cancer. In one embodiment, the cancer is a primary tumor. In some embodiments, the subject is a mammal. In some embodiments, the subject is a human.

In some embodiments, the CD36 inhibitor is an antibody, a single chain antibody, or a scFv, Fab or F(ab′)2 fragment. In one embodiment, the CD36 inhibitor is an antibody. In an embodiment, the CD36 inhibitor is a humanized antibody. In certain embodiments, the CD36 inhibitor is the antibody JC63.1. In one embodiment, the CD36 inhibitor is a shRNA or an iRNA, a siRNA, or an antisense RNA or DNA.

In some embodiments, the second therapy is an immunotherapy. In one embodiment, the immunotherapy is a PD-1 inhibitor. In an embodiment, the PD-1 inhibitor is an anti-PD-1 antibody. In one embodiment, the anti-PD-1 antibody is pembrolizumab (KEYTRUDA; MK-3475), pidilizumab (CT-011), or nivolumab (OPDIVO; BMS-936558). In an embodiment, the immunotherapy is a PD-L1 inhibitor. In one embodiment, PD-L1 inhibitor is an anti-PD-L1 antibody. In an embodiment, the anti-PD-L1 antibody is atezolizumab (Tecentriq or RG7446), durvalumab (Imfinzi or MEDI4736), avelumab (Bavencio) or BMS-936559 In one embodiment, the immunotherapy is a CTLA-4 inhibitor. In an embodiment, the CTLA-4 inhibitor is an anti-CTLA-4 antibody. In one embodiment, the anti-CTLA-4 antibody is ipilimumab or an antigen-binding fragment thereof.

In one embodiment, the second therapy is a chemotherapy, such as a chemotherapeutic agent. In an embodiment, the chemotherapeutic agent is cisplatin. In certain embodiments, the chemotherapeutic agent comprises one of the anti-cancer drugs or anti-cancer drug combinations listed in Table A.

TABLE A

| | | | |
|---|---|---|---|
| Abemaciclib | Abiraterone | Abraxane (Paclitaxel | ABVD |
| | Acetate | Albumin-stabilized | |
| | | Nanoparticle | |
| | | Formulation) | |
| ABVE | ABVE-PC | AC | Acalabrutinib |
| AC-T | Actemra | Adcetris (Brentuximab | ADE |
| | (Tocilizumab) | Vedotin) | |
| Ado-Trastuzumab | Adriamycin | Afatinib Dimaleate | Afmitor |
| Emtansine | (Doxorubicin | | (Everolimus) |
| | Hydrochloride) | | |
| Akynzeo | Aldara | Aldesleukin | Alecensa |
| (Netupitant and | (Imiquimod) | | (Alectinib) |
| Palonosetron | | | |
| Hydrochloride) | | | |
| Alectinib | Alemtuzumab | Alimta (Pemetrexed | Aliqopa |
| | | Disodium) | (Copanlisib |
| | | | Hydrochloride) |
| Alkeran for | Alkeran Tablets | Aloxi (Palonosetron | Alunbrig |
| Injection | (Melphalan) | Hydrochloride) | (Brigatinib) |
| (Melphalan | | | |
| Hydrochloride) | | | |
| Ameluz | Amifostine | Aminolevulinic Acid | Anastrozole |
| (Aminolevulinic | | | |
| Acid) | | | |
| Apalutamide | Aprepitant | Aranesp (Darbepoetin | Aredia |
| | | Alfa) | (Pamidronate |
| | | | Disodium) |
| Arimidex | Aromasin | Arranon (Nelarabine) | Arsenic Trioxide |
| (Anastrozole) | (Exemestane) | | |
| Arzerra | Asparaginase | Atezolizumab | Avastin |
| (Ofatumumab) | Erwinia | | (Bevacizumab) |
| | chrysanthemi | | |
| Avelumab | Axicabtagene | Axitinib | Azacitidine |
| | Ciloleucel | | |
| Azedra | Bavencio | BEACOPP | Beleodaq |
| (Iobenguane I 131) | (Avelumab) | | (Belinostat) |
| Belinostat | Bendamustine | Bendeka (Bendamustine | BEP |
| | Hydrochloride | Hydrochloride) | |
| Besponsa | Bevacizumab | Bexarotene | Bicalutamide |
| (Inotuzumab | | | |
| Ozogamicin) | | | |
| BiCNU | Binimetinib | Bleomycin | Blinatumomab |
| (Carmustine) | | | |
| Blincyto | Bortezomib | Bosulif (Bosutinib) | Bosutinib |
| (Blinatumomab) | | | |
| Braftovi | Brentuximab | Brigatinib | BuMel |
| (Encorafenib) | Vedotin | | |
| Busulfan | Busulfex | Cabazitaxel | Cabometyx |
| | (Busulfan) | | (Cabozantinib-S- |
| | | | Malate) |
| Cabozantinib-S- | CAF | Calquence | Campath |
| Malate | | (Acalabrutinib) | (Alemtuzumab) |
| Camptosar | Capecitabine | CAPOX | Carac |
| (Irinotecan | | | (Fluorouracil-- |
| Hydrochloride) | | | Topical) |
| Carboplatin | CARBOPLATIN- | Carfilzomib | Carmustine |
| | TAXOL | | |
| Carmustine | Casodex | CEM | Cemiplimab-rwlc |
| Implant | (Bicalutamide) | | |
| Ceritinib | Cerubidine | Cervarix (Recombinant | Cetuximab |
| | (Daunorubicin | HPV Bivalent Vaccine) | |
| | Hydrochloride) | | |
| CEV | Chlorambucil | CHLORAMBUCIL- | CHOP |
| | | PREDNISONE | |
| Cisplatin | Cladribine | Clofarabine | Clolar |
| | | | (Clofarabine) |
| CMF | Cobimetinib | Cometriq (Cabozantinib- | Copanlisib |
| | | S-Malate) | Hydrochloride |
| COPDAC | Copiktra | COPP | COPP-ABV |
| | (Duvelisib) | | |
| Cosmegen | Cotellic | Crizotinib | CVP |
| (Dactinomycin) | (Cobimetinib) | | |
| Cyclophosphamide | Cyramza | Cytarabine | Cytarabine |
| | (Ramucirumab) | | Liposome |
| Cytosar-U | Dabrafenib | Dacarbazine | Dacogen |
| (Cytarabine) | | | (Decitabine) |
| Dacomitinib | Dactinomycin | Daratumumab | Darbepoetin Alfa |
| Darzalex | Dasatinib | Daunorubicin | Daunorubicin |
| (Daratumumab) | | Hydrochloride | Hydrochloride |
| | | | and Cytarabine |
| | | | Liposome |
| Decitabine | Defibrotide | Defitelio (Defibrotide | Degarelix |
| | Sodium | Sodium) | |
| Denileukin | Denosumab | DepoCyt (Cytarabine | Dexamethasone |
| Diftitox | | Liposome) | |
| Dexrazoxane | Dinutuximab | Docetaxel | Doxil |
| Hydrochloride | | | (Doxorubicin |
| | | | Hydrochloride |
| | | | Liposome) |
| Doxorubicin | Doxorubicin | Dox-SL (Doxorubicin | Durvalumab |
| Hydrochloride | Hydrochloride | Hydrochloride | |
| | Liposome | Liposome) | |
| Duvelisib | Efudex | Eligard (Leuprolide | Elitek |
| | (Fluorouracil-- | Acetate) | (Rasburicase) |
| | Topical) | | |
| Ellence | Elotuzumab | Eloxatin (Oxaliplatin) | Eltrombopag |
| (Epirubicin | | | Olamine |
| Hydrochloride) | | | |
| Emend | Empliciti | Enasidenib Mesylate | Encorafenib |
| (Aprepitant) | (Elotuzumab) | | |
| Enzalutamide | Epirubicin | EPOCH | Epoetin Alfa |
| | Hydrochloride | | |
| Epogen | Erbitux | Eribulin Mesylate | Erivedge |
| (Epoetin Alfa) | (Cetuximab) | | (Vismodegib) |
| Erleada | Erlotinib | Erwinaze (Asparaginase | Ethyol |
| (Apalutamide) | Hydrochloride | Erwinia chrysanthemi | (Amifostine) |
| Etopophos | Etoposide | Etoposide Phosphate | Evacet |
| (Etoposide | | | (Doxorubicin |
| Phosphate) | | | Hydrochloride |
| | | | Liposome) |
| Everolimus | Evista (Raloxifene | Evomela (Melphalan | Exemestane |
| | Hydrochloride) | Hydrochloride) | |
| 5-FU (Fluorouracil | 5-FU | Fareston (Toremifene) | Farydak |
| Injection) | (Fluorouracil-- | | (Panobinostat) |
| | Topical) | | |
| Faslodex | FEC | Femara (Letrozole) | Filgrastim |
| (Fulvestrant) | | | |
| Firmagon | Fludarabine | Fluoroplex (Fluorouracil-- | Fluorouracil |
| (Degarelix) | Phosphate | Topical) | Injection |
| Fluorouracil-- | Flutamide | FOLFIRI | FOLFIRI- |
| Topical | | | BEVACIZUMAB |
| FOLFIRI- | FOLFIRINOX | FOLFOX | Folotyn |
| CETUXIMAB | | | (Pralatrexate) |
| Fostamatinib | FU-LV | Fulvestrant | Fusilev |
| Disodium | | | (Leucovorin |
| | | | Calcium) |
| Gardasil | Gardasil 9 | Gazyva (Obinutuzumab) | Gefitinib |
| (Recombinant | (Recombinant | | |
| HPV Quadrivalent | HPV Nonavalent | | |
| Vaccine) | Vaccine) | | |
| Gemcitabine | GEMCITABINE- | GEMCITABINE- | Gemtuzumab |
| Hydrochloride | CISPLATIN | OXALIPLATIN | Ozogamicin |
| Gemzar | Gilotrif (Afatinib | Gleevec (Imatinib | Gliadel Wafer |
| (Gemcitabine | Dimaleate) | Mesylate) | (Carmustine |
| Hydrochloride) | | | Implant) |
| Glucarpidase | Goserelin Acetate | Granisetron | Granisetron |
| | | | Hydrochloride |
| Granix | Halaven (Eribulin | Hemangeol (Propranolol | Herceptin |
| (Filgrastim) | Mesylate) | Hydrochloride) | (Trastuzumab) |
| HPV Bivalent | HPV Nonavalent | HPV Quadrivalent | Hycamtin |
| Vaccine, | Vaccine, | Vaccine, | (Topotecan |
| Recombinant | Recombinant | Recombinant | Hydrochloride) |
| Hydrea | Hydroxyurea | Hyper-CVAD | Ibrance |
| (Hydroxyurea) | | | (Palbociclib) |
| Ibritumomab | Ibrutinib | ICE | Iclusig (Ponatinib |
| Tiuxetan | | | Hydrochloride) |
| Idarubicin | Idelalisib | Idhifa (Enasidenib | Ifex (Ifosfamide) |
| Hydrochloride | | Mesylate) | |
| Ifosfamide | IL-2 | Imatinib Mesylate | Imbruvica |
| | (Aldesleukin) | | (Ibrutinib) |
| Imfinzi | Imiquimod | Imlygic (Talimogene | Inlyta (Axitinib) |
| (Durvalumab) | | Laherparepvec) | |
| Inotuzumab | Interferon Alfa- | Interleukin-2 | Intron A |
| Ozogamicin | 2b, Recombinant | (Aldesleukin) | (Recombinant |
| | | | Interferon Alfa- |
| | | | 2b) |
| Iobenguane I 131 | Ipilimumab | Iressa (Gefitinib) | Irinotecan |
| | | | Hydrochloride |
| Irinotecan | Istodax | Ivosidenib | Ixabepilone |
| Hydrochloride | (Romidepsin) | | |
| Liposome | | | |
| Ixazomib Citrate | Ixempra | Jakafi (Ruxolitinib | JEB |
| | (Ixabepilone) | Phosphate) | |
| Jevtana | Kadcyla (Ado- | Kepivance (Palifermin) | Keytruda |
| (Cabazitaxel) | Trastuzumab | | (Pembrolizumab) |
| | Emtansine) | | |
| Kisqali | Kymriah | Kyprolis (Carfilzomib) | Lanreotide |
| (Ribociclib) | (Tisagenlecleucel) | | Acetate |
| Lapatinib | Larotrectinib | Lartruvo (Olaratumab) | Lenalidomide |
| Ditosylate | Sulfate | | |
| Lenvatinib | Lenvima | Letrozole | Leucovorin |
| Mesylate | (Lenvatinib | | Calcium |
| | Mesylate) | | |
| Leukeran | Leuprolide | Levulan | Libtayo |
| (Chlorambucil) | Acetate | Kerastik (Aminolevulinic | (Cemiplimab- |
| | | Acid) | rwlc) |
| LipoDox | Lomustine | Lonsurf (Trifluridine and | Lorbrena |
| (Doxorubicin | | Tipiracil Hydrochloride) | (Lorlatinib) |
| Hydrochloride | | | |
| Liposome) | | | |
| Lorlatinib | Lumoxiti | Lupron (Leuprolide | Lupron Depot |
| | (Moxetumomab | Acetate) | (Leuprolide |
| | Pasudotox-tdfk) | | Acetate) |
| Lutathera | Lutetium (Lu 177- | Lynparza (Olaparib) | Marqibo |
| (Lutetium Lu 177- | Dotatate) | | (Vincristine |
| Dotatate) | | | Sulfate |
| | | | Liposome) |
| Matulane | Mechlorethamine | Megestrol Acetate | Mekinist |
| (Procarbazine | Hydrochloride | | (Trametinib) |
| Hydrochloride) | | | |
| Mektovi | Melphalan | Melphalan | Mercaptopurine |
| (Binimetinib) | | Hydrochloride | |
| Mesna | Mesnex (Mesna) | Methotrexate | Methylnaltrexone |
| | | | Bromide |
| Midostaurin | Mitomycin C | Mitoxantrone | Mogamulizumab- |
| | | Hydrochloride | kpkc |
| Moxetumomab | Mozobil | Mustargen | MVAC |
| Pasudotox-tdfk | (Plerixafor) | (Mechlorethamine | |
| | | Hydrochloride) | |
| Myleran | Mylotarg | Nanoparticle Paclitaxel | Navelbine |
| (Busulfan) | (Gemtuzumab | (Paclitaxel Albumin- | (Vinorelbine |
| | Ozogamicin) | stabilized Nanoparticle | Tartrate) |
| | | Formulation) | |
| Necitumumab | Nelarabine | Neratinib Maleate | Nerlynx |
| | | | (Neratinib |
| | | | Maleate) |
| Netupitant and | Neulasta | Neupogen (Filgrastim) | Nexavar |
| Palonosetron | (Pegfilgrastim) | | (Sorafenib |
| Hydrochloride | | | Tosylate) |
| Nilandron | Nilotinib | Nilutamide | Ninlaro |
| (Nilutamide) | | | (Ixazomib |
| | | | Citrate) |
| Niraparib | Nivolumab | Nplate (Romiplostim) | Obinutuzumab |
| Tosylate | | | |
| Monohydrate | | | |
| Odomzo | OEPA | Ofatumumab | OFF |
| (Sonidegib) | | | |
| Olaparib | Olaratumab | Omacetaxine | Oncaspar |
| | | Mepesuccinate | (Pegaspargase) |
| Ondansetron | Onivyde | Ontak (Denileukin | Opdivo |
| Hydrochloride | (Irinotecan | Diftitox) | (Nivolumab) |
| | Hydrochloride | | |
| | Liposome) | | |
| OPPA | Osimertinib | Oxaliplatin | Paclitaxel |
| Paclitaxel | PAD | Palbociclib | Palifermin |
| Albumin- | | | |
| stabilized | | | |
| Nanoparticle | | | |
| Formulation | | | |
| Palonosetron | Palonosetron | Pamidronate Disodium | Panitumumab |
| Hydrochloride | Hydrochloride | | |
| | and Netupitant | | |
| Panobinostat | Pazopanib | PCV | PEB |
| | Hydrochloride | | |
| Pegaspargase | Pegfilgrastim | Peginterferon Alfa-2b | PEG-Intron |
| | | | (Peginterferon |
| | | | Alfa-2b) |
| Pembrolizumab | Pemetrexed | Perjeta (Pertuzumab) | Pertuzumab |
| | Disodium | | |
| Plerixafor | Pomalidomide | Pomalyst | Ponatinib |
| | | (Pomalidomide) | Hydrochloride |
| Portrazza | Poteligeo | Pralatrexate | Prednisone |
| (Necitumumab) | (Mogamulizumab- | | |
| | kpkc) | | |
| Procarbazine | Procrit (Epoetin | Proleukin (Aldesleukin) | Prolia |
| Hydrochloride | Alfa) | | (Denosumab) |
| Promacta | Propranolol | Provenge (Sipuleucel-T) | Purinethol |
| (Eltrombopag | Hydrochloride | | (Mercaptopurine) |
| Olamine) | | | |
| Purixan | Radium 223 | Raloxifene | Ramucirumab |
| (Mercaptopurine) | Dichloride | Hydrochloride | |
| Rasburicase | R-CHOP | R-CVP | Recombinant |
| | | | Human |
| | | | Papillomavirus |
| | | | (HPV) Bivalent |
| | | | Vaccine |
| Recombinant | Recombinant | Recombinant Interferon | Regorafenib |
| Human | Human | Alfa-2b | |
| Papillomavirus | Papillomavirus | | |
| (HPV) Nonavalent | (HPV) | | |
| Vaccine | Quadrivalent | | |
| | Vaccine | | |
| Relistor | R-EPOCH | Retacrit (Epoetin Alfa) | Revlimid |
| (Methylnaltrexone | | | (Lenalidomide) |
| Bromide) | | | |
| Rheumatrex | Ribociclib | R-ICE | Rituxan |
| (Methotrexate) | | | (Rituximab) |
| Rituxan Hycela | Rituximab | Rituximab and | Rolapitant |
| (Rituximab and | | Hyaluronidase Human | Hydrochloride |
| Hyaluronidase | | | |
| Human) | | | |
| Romidepsin | Romiplostim | Rubidomycin | Rubraca |
| | | (Daunorubicin | (Rucaparib |
| | | Hydrochloride) | Camsylate) |
| Rucaparib | Ruxolitinib | Rydapt (Midostaurin) | Sancuso |
| Camsylate | Phosphate | | (Granisetron) |
| Sclerosol | Siltuximab | Sipuleucel-T | Somatuline Depot |
| Intrapleural | | | (Lanreotide |
| Aerosol (Talc) | | | Acetate) |
| Sonidegib | Sorafenib | Sprycel (Dasatinib) | STANFORD V |
| | Tosylate | | |
| Sterile Talc | Steritalc | Stivarga (Regorafenib) | Sunitinib Malate |
| Powder (Talc) | (Talc) | | |
| Sustol | Sutent | Sylatron (Peginterferon | Sylvant |
| (Granisetron) | (Sunitinib | Alfa-2b) | (Siltuximab) |
| | Malate) | | |
| Synribo | Tabloid | TAC | Tafinlar |
| (Omacetaxine | (Thioguanine) | | (Dabrafenib) |
| Mepesuccinate) | | | |
| Tagrisso | Talc | Talimogene | Tamoxifen |
| (Osimertinib) | | Laherparepvec | Citrate |
| Tarabine PFS | Tarceva (Erlotinib | Targretin (Bexarotene) | Tasigna |
| (Cytarabine) | Hydrochloride) | | (Nilotinib) |
| Tavalisse | Taxol (Paclitaxel) | Taxotere (Docetaxel) | Tecentriq |
| (Fostamatinib | | | (Atezolizumab) |
| Disodium) | | | |
| Temodar | Temozolomide | Temsirolimus | Thalidomide |
| (Temozolomide) | | | |
| Thalomid | Thioguanine | Thiotepa | Tibsovo |
| (Thalidomide) | | | (Ivosidenib) |
| Tisagenlecleucel | Tocilizumab | Tolak (Fluorouracil-- | Topotecan |
| | | Topical) | Hydrochloride |
| Toremifene | Torisel | Totect (Dexrazoxane | TPF |
| | (Temsirolimus) | Hydrochloride) | |
| Trabectedin | Trametinib | Trastuzumab | Treanda |
| | | | (Bendamustine |
| | | | Hydrochloride) |
| Trexall | Trifluridine and | Trisenox (Arsenic | Tykerb (Lapatinib |
| (Methotrexate) | Tipiracil | Trioxide) | Ditosylate) |
| | Hydrochloride | | |
| Unituxin | Uridine Triacetate | VAC | Valrubicin |
| (Dinutuximab) | | | |
| Valstar | Vandetanib | VAMP | Varubi |
| (Valrubicin) | | | (Rolapitant |
| | | | Hydrochloride) |
| Vectibix | VeIP | Velcade (Bortezomib) | Vemurafenib |
| (Panitumumab) | | | |
| Venclexta | Venetoclax | Verzenio (Abemaciclib) | Vidaza |
| (Venetoclax) | | | (Azacitidine) |
| Vinblastine | Vincristine | Vincristine Sulfate | Vinorelbine |
| Sulfate | Sulfate | Liposome | Tartrate |
| VIP | Vismodegib | Vistogard | Vitrakvi |
| | | (Uridine | (Larotrectinib |
| | | Triacetate) | Sulfate) |
| Vizimpro | Voraxaze | Vorinostat | Votrient |
| (Dacomitinib) | (Glucarpidase) | | (Pazopanib |
| | | | Hydrochloride) |
| Vyxeos | Xalkori | Xeloda (Capecitabine) | XELIRI |
| (Daunorubicin | (Crizotinib) | | |
| Hydrochloride and | | | |
| Cytarabine | | | |
| Liposome) | | | |
| XELOX | Xgeva | Xofigo (Radium 223 | Xtandi |
| | (Denosumab) | Dichloride) | (Enzalutamide) |
| Yervoy | Yescarta | Yondelis (Trabectedin) | Zaltrap (Ziv- |
| (Ipilimumab) | (Axicabtagene | | Aflibercept) |
| | Ciloleucel) | | |
| Zarxio (Filgrastim) | Zejula (Niraparib | Zelboraf (Vemurafenib) | Zevalin |
| | Tosylate | | (Ibritumomab |
| | Monohydrate) | | Tiuxetan) |
| Zinecard | Ziv-Aflibercept | Zofran (Ondansetron | Zoladex |
| (Dexrazoxane | | Hydrochloride) | (Goserelin |
| Hydrochloride) | | | Acetate) |
| Zoledronic Acid | Zolinza | Zometa (Zoledronic | Zydelig |
| | (Vorinostat) | Acid) | (Idelalisib) |
| Zykadia | Zytiga | | |
| (Ceritinib) | (Abiraterone | | |
| | Acetate) | | |

In some embodiments, the present invention provides methods of treating cancer in a mammal using a combination of a CD36 inhibitor and anti-PD-1 antibody. In some embodiments, the cancer is selected from the group consisting of: oral squamous cell carcinoma, head and neck cancer, esophageal cancer, gastric cancer, ovarian cancer, cervical cancer, lung cancer, breast cancer, colon cancer, renal cancer, prostate cancer, sarcoma, melanoma, leukemia, and lymphoma. In some embodiments, the cancer is oral squamous cell carcinoma. In some embodiments, the cancer is ovarian cancer. In some embodiments, the cancer is colon cancer. In some embodiments, the cancer is lung cancer. In some embodiments, the cancer is melanoma. In a further embodiment, the cancer is any other cancer disclosed herein. In one embodiment, the cancer is metastatic cancer. In one embodiment, the cancer is a primary tumor. In embodiments, the CD36 inhibitor is an antibody, a single chain antibody, or a scFv, Fab or F(ab′)2 fragment. In one embodiment, the CD36 inhibitor is an antibody. In an embodiment, the CD36 inhibitor is a humanized antibody. In certain embodiments, the CD36 inhibitor is the antibody JC63.1. In one embodiment, the CD36 inhibitor is a shRNA or an iRNA, a siRNA, or an antisense RNA or DNA. In one embodiment, the anti-PD-1 antibody is pembrolizumab (KEYTRUDA; MK-3475), pidilizumab (CT-011), or nivolumab (OPDIVO; BMS-936558).

Examples of cancers and/or malignant tumors that may be treated using the methods of the invention, include liver cancer, hepatocellular carcinoma (HCC), bone cancer, pancreatic cancer, skin cancer, oral cancer, cancer of the head or neck, breast cancer, lung cancer, small cell lung cancer, NSCLC, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, squamous cell carcinoma of the head and neck (SCCHN), non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, hematologic malignancies including, for example, multiple myeloma, B-cell lymphoma, Hodgkin lymphoma/primary mediastinal B-cell lymphoma, non-Hodgkin's lymphomas, acute myeloid lymphoma, chronic myelogenous leukemia, chronic lymphoid leukemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T-cell lymphoma, and precursor T-lymphoblastic lymphoma, and any combinations of said cancers. The present invention is applicable to treatment of both primary tumors and metastatic tumors. In some embodiments, the cancer is oral squamous cell carcinoma. In some embodiments, the cancer is ovarian cancer. In some embodiments, the cancer is colon cancer. In some embodiments, the cancer is lung cancer. In some embodiments, the cancer is melanoma.

In some embodiments, the present invention provides methods that reduce the number of metastases in a subject. In some embodiments, the methods reduce the number of metastases by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% in a subject. In some embodiments, the methods reduce the number of metastases by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% in a mouse model of cancer, relative to control untreated mice.

In some embodiments, the present invention provides methods that reduce the size of metastases in a subject. In some embodiments, the methods reduce the size of metastases by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% in a subject. In some embodiments, the methods reduce the size of metastases by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% in a mouse model of cancer, relative to control untreated mice. In some embodiments, the size is reduced as measured by IVIS imaging or H&E staining.

In some embodiments, the present invention provides methods that inhibit the growth of one or more tumors in a subject. In some embodiments, the methods inhibit the growth of one or more tumors by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% in a subject. In some embodiments, the methods inhibit the growth of one or more tumors by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% in a mouse model of cancer, relative to untreated controls. In some of these embodiments, the one or more tumors are metastatic tumors.

In some embodiments, the present invention provides methods that increase the amount of necrosis in one or more tumors. In some embodiments, the methods result in an increase of necrosis of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% in a subject's tumors. In some embodiments, the methods result in an increase of necrosis of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% in a tumors in a mouse model of cancer, relative to untreated controls.

In some embodiments, the present invention provides methods that increase the amount of fibrosis in one or more tumors. In some embodiments, the methods result in an increase of fibrosis of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% in a subject's tumors. In some embodiments, the methods result in an increase of fibrosis of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% in a tumors in a mouse model of cancer, relative to untreated controls.

In some embodiments, the present invention provides methods that increase both the necrosis and fibrosis in one or more tumors in a subject. In some embodiments, the present invention provides methods that increase both the necrosis and fibrosis in one or more tumors in a mouse model of cancer, relative to untreated controls.

In embodiments, the antibodies can be administered systemically, for instance, intraperitoneally, and can be in the form of an appropriate suspension, for instance an aqueous suspension, in water or another appropriate liquid such as saline solution.

For administration of the antibodies, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. In certain embodiments, the antibodies are administered at a flat or fixed dose. In embodiments, the antibodies are administered at any dosage described for the antibody in the art.

### Anti-PD-1 and Anti-PD-L1 Antibodies

As used herein, the terms “Programmed Death 1,” “Programmed Cell Death 1,” “Protein PD-1,” “PD-1,” “PD1,” “PDCD1,” “hPD-1” and “hPD-I” are used interchangeably, and include variants, isoforms, species homologs of human PD-1, and analogs having at least one common epitope with PD-1. The complete PD-1 sequence can be found under GenBank Accession No. U64863.

The protein Programmed Death 1 (PD-1) is an inhibitory member of the CD28 family of receptors, that also includes CD28, CTLA-4, ICOS and BTLA. PD-1 is expressed on activated B cells, T cells, and myeloid cells (Agata et al., supra; Okazaki et al. (2002) Curr. Opin. Immunol. 14: 391779-82; Bennett et al. (2003) J Immunol 170:711-8). The initial members of the family, CD28 and ICOS, were discovered by functional effects on augmenting T cell proliferation following the addition of monoclonal antibodies (Hutloff et al. Nature (1999); 397:263-266; Hansen et al. Immunogenics (1980); 10:247-260). PD-1 was discovered through screening for differential expression in apoptotic cells (Ishida et al. EMBO J (1992); 11:3887-95). The other members of the family, CTLA-4 and BTLA, were discovered through screening for differential expression in cytotoxic T lymphocytes and TH1 cells, respectively. CD28, ICOS and CTLA-4 all have an unpaired cysteine residue allowing for homodimerization. In contrast, PD-1 is suggested to exist as a monomer, lacking the unpaired cysteine residue characteristic in other CD28 family members.

The PD-1 gene is a 55 kDa type I transmembrane protein that is part of the Ig gene superfamily (Agata et al. (1996) Int Immunol 8:765-72). PD-1 contains a membrane proximal immunoreceptor tyrosine inhibitory motif (ITIM) and a membrane distal tyrosine-based switch motif (ITSM) (Thomas, M. L. (1995) J Exp Med 181:1953-6; Vivier, E and Daeron, M (1997) Immunol Today 18:286-91). Although structurally similar to CTLA-4, PD-1 lacks the MYPPPY motif (SEQ ID NO: 32) that is critical for B7-1 and B7-2 binding. Two ligands for PD-1 have been identified, PD-L1 and PD-L2, that have been shown to downregulate T cell activation upon binding to PD-1 (Freeman et al. (2000) J Exp Med 192:1027-34; Latchman et al. (2001) Nat Immunol 2:261-8; Carter et al. (2002) Eur J Immunol 32:634-43). Both PD-L1 and PD-L2 are B7 homologs that bind to PD-1, but do not bind to other CD28 family members. PD-L1 is abundant in a variety of human cancers (Dong et al. (2002) Nat. Med. 8:787-9). The interaction between PD-1 and PD-L1 results in a decrease in tumor infiltrating lymphocytes, a decrease in T-cell receptor mediated proliferation, and immune evasion by the cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1, and the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66).

Consistent with PD-1 being an inhibitory member of the CD28 family, PD-1 deficient animals develop various autoimmune phenotypes, including autoimmune cardiomyopathy and a lupus-like syndrome with arthritis and nephritis (Nishimura et al. (1999) Immunity 11:141-51; Nishimura et al. (2001) Science 291:319-22). Additionally, PD-1 has been found to play a role in autoimmune encephalomyelitis, systemic lupus erythematosus, graft-versus-host disease (GVHD), type I diabetes, and rheumatoid arthritis (Salama et al. (2003) J Exp Med 198:71-78; Prokunina and Alarcon-Riquelme (2004) Hum Mol Genet 13:R143; Nielsen et al. (2004) Lupus 13:510). In a murine B cell tumor line, the ITSM of PD-1 was shown to be essential to block BCR-mediated Ca. sup.2+-flux and tyrosine phosphorylation of downstream effector molecules (Okazaki et al. (2001) PNAS 98:13866-71).

“Programmed Death Ligand-1 (PD-L1)” is one of two cell surface glycoprotein ligands for PD-1 (the other being PD-L2) that down-regulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

Some embodiments of the invention include an anti-PD-1 antibody, or an anti-PD-L1 antibody, or antigen binding fragments thereof in combination with a CD36 inhibitor such as an anti-CD36 antibody or antigen binding fragment thereof. PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to down regulate T cell activation and cytokine secretion upon binding to PD-1. Inhibition of the PD-1/PD-L1 interaction mediates potent antitumor activity in preclinical models.

Human monoclonal antibodies (HuMAbs) that bind specifically to PD-1 with high affinity have been disclosed in U.S. Pat. Nos. 8,008,449 and 8,779,105—both of which are incorporated herein by reference in their entirety. Other anti-PD-1 mAbs have been described in, for example, U.S. Pat. Nos. 6,808,710, 7,488,802, 8,168,757 and 8,354,509, and PCT Publication Nos. WO2012/145493 and WO2016/168716—each of which is incorporated herein by reference in its entirety. Each of the anti-PD-1 HuMAbs disclosed in U.S. Pat. No. 8,008,449 has been demonstrated to exhibit one or more of the following characteristics: (a) binds to human PD-1 with a KD of 1×10-7 M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates Ab responses; and (j) inhibits tumor cell growth in vivo. Anti-PD-1 antibodies useful for the present invention include mAbs that bind specifically to human PD-1 and exhibit at least one, preferably at least five, of the preceding characteristics.

Anti-human-PD-1 antibodies (or VH and/or VL domains derived therefrom) suitable for use in the invention can be generated using methods well known in the art. Alternatively, art recognized anti-PD-1 antibodies can be used. For example, monoclonal antibodies 5C4 (referred to herein as Nivolumab or BMS-936558), 17D8, 2D3, 4H1, 4A11, 7D3, and 5F4, described in WO 2006/121168, the teachings of which are hereby incorporated by reference, can be used. Other known PD-1 antibodies include lambrolizumab (MK-3475) described in WO 2008/156712, and AMP-514 described in WO 2012/145493. Further known anti-PD-1 antibodies and other PD-1 inhibitors include those described in WO 2009/014708, WO 03/099196, WO 2009/114335 and WO 2011/161699. Another known anti-PD-1 antibody is pidilizumab (CT-011). Antibodies or antigen binding fragments thereof that compete with any of these antibodies or inhibitors for binding to PD-1 also can be used.

In one embodiment, the anti-PD-1 antibody is nivolumab. Nivolumab (also known as “OPDIVO®”; BMS-936558; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56). In another embodiment, the anti-PD-1 antibody or fragment thereof cross-competes with nivolumab. In other embodiments, the anti-PD-1 antibody or fragment thereof binds to the same epitope as nivolumab. In certain embodiments, the anti-PD-1 antibody has the same CDRs as nivolumab.

In another embodiment, the anti-PD-1 antibody is pembrolizumab. Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587.

In another embodiment, the anti-PD-1 antibody or antigen binding fragment thereof cross-competes with pembrolizumab. In some embodiments, the anti-PD-1 antibody or antigen binding fragment thereof binds to the same epitope as pembrolizumab. In certain embodiments, the anti-PD-1 antibody or antigen binding fragment thereof has the same CDRs as pembrolizumab. In another embodiment, the anti-PD-1 antibody is pembrolizumab. Pembrolizumab (also known as “KEYTRUDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587; see also http://www.cancer.gov/drugdictionary?cdrid=695789 (last accessed: May 25, 2017). Pembrolizumab has been approved by the FDA for the treatment of relapsed or refractory melanoma.

In other embodiments, the anti-PD-1 antibody or antigen binding fragment thereof cross-competes with MEDI0608. In still other embodiments, the anti-PD-1 antibody or antigen binding fragment thereof binds to the same epitope as MEDI0608. In certain embodiments, the anti-PD-1 antibody has the same CDRs as MEDI0608. In other embodiments, the anti-PD-1 antibody is MEDI0608 (formerly AMP-514), which is a monoclonal antibody. MEDI0608 is described, for example, in U.S. Pat. No. 8,609,089 or in http://www.cancer.gov/drugdictionary?cdrid=756047 (last accessed May 25, 2017).

In other embodiments, the anti-PD-1 antibody or antigen binding fragment thereof cross-competes with BGB-A317. In some embodiments, the anti-PD-1 antibody or antigen binding fragment thereof binds the same epitope as BGB-A317. In certain embodiments, the anti-PD-1 antibody or antigen binding fragment thereof has the same CDRs as BGB-A317. In certain embodiments, the anti-PD-1 antibody or antigen binding fragment thereof is BGB-A317, which is a humanized monoclonal antibody. BGB-A317 is described in U.S. Publ. No. 2015/0079109.

Anti-PD-1 antibodies useful for the disclosed compositions also include isolated antibodies that bind specifically to human PD-1 and cross-compete for binding to human PD-1 with nivolumab (see, e.g., U.S. Pat. Nos. 8,008,449 and 8,779,105; Int'l Pub. No. WO 2013/173223). The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar to those of nivolumab by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., Int'l Pub. No. WO 2013/173223).

In certain embodiments, antibodies or antigen binding fragments thereof that cross-compete for binding to human PD-1 with, or bind to the same epitope region of human PD-1 as, nivolumab are mAbs. For administration to human subjects, these cross-competing antibodies can be chimeric antibodies, or humanized or human antibodies. Such chimeric, humanized or human mAbs can be prepared and isolated by methods well known in the art.

Anti-PD-1 antibodies useful for the compositions of the disclosed invention also include antigen-binding portions of the above antibodies. It has been amply demonstrated that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; and (iv) a Fv fragment consisting of the VL and VH domains of a single arm of an antibody.

Anti-PD-1 antibodies suitable for use in the disclosed compositions are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and upregulating the immune system. In certain embodiments, the anti-PD-1 antibody or antigen-binding portion thereof cross-competes with nivolumab for binding to human PD-1. In other embodiments, the anti-PD-1 antibody or antigen-binding portion thereof is a chimeric, humanized or human monoclonal antibody or a portion thereof. In certain embodiments, the antibody is a humanized antibody. In other embodiments, the antibody is a human antibody. Antibodies of an IgG1, IgG2, IgG3 or IgG4 isotype can be used.

In certain embodiments, the anti-PD-1 antibody or antigen binding fragment thereof comprises a heavy chain constant region which is of a human IgG1 or IgG4 isotype. In certain other embodiments, the sequence of the IgG4 heavy chain constant region of the anti-PD-1 antibody or antigen binding fragment thereof contains an S228P mutation which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies (Wang et al., 2014). In yet other embodiments, the antibody comprises a light chain constant region which is a human kappa or lambda constant region. In other embodiments, the anti-PD-1 antibody or antigen binding fragment thereof is a mAb or an antigen-binding portion thereof. In certain embodiments of any of the therapeutic methods described herein comprising administration of an anti-PD-1 antibody, the anti-PD-1 antibody is nivolumab. In other embodiments, the anti-PD-1 antibody is pembrolizumab. In other embodiments, the anti-PD-1 antibody is chosen from the human antibodies 17D8, 2D3, 4H1, 4A11, 7D3 and 5F4 described in U.S. Pat. No. 8,008,449. In still other embodiments, the anti-PD-1 antibody is MEDI0608 (formerly AMP-514), AMP-224, or Pidilizumab (CT-011). Other known PD-1 antibodies include lambrolizumab (MK-3475) described in, for example, WO 2008/156712, and AMP-514 described in, for example, WO 2012/145493. Further known anti-PD-1 antibodies and other PD-1 inhibitors include those described in, for example, WO 2009/014708, WO 03/099196, WO 2009/114335 and WO 2011/161699. In one embodiment, the anti-PD-1 antibody is REGN2810. In one embodiment, the anti-PD-1 antibody is PDR001. Another known anti-PD-1 antibody is pidilizumab (CT-011). Each of the above references are incorporated by reference. Antibodies or antigen binding fragments thereof that compete with any of these antibodies or inhibitors for binding to PD-1 also can be used.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540, each of which are herein incorporated by reference.

In some embodiments, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; see Si-Yang Liu et al., J. Hematol. Oncol. 10:136 (2017)), BGB-A317 (Beigene; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), and IBI308 (Innovent; see WO 2017/024465, WO 2017/025016, WO 2017/132825, and WO 2017/133540). Each of the above references are herein incorporated by reference.

In embodiments, the anti-PD-1 antibody is a bispecific antibody. In embodiments, the second therapy is a PD-1 inhibitor. In embodiments, the PD-1 inhibitor is a small molecule.

Because anti-PD-1 antibodies and anti-PD-L1 antibodies target the same signaling pathway and have been shown in clinical trials to exhibit similar levels of efficacy in a variety of cancers, an anti-PD-L1 antibody or antigen binding fragment thereof can be substituted for an anti-PD-1 antibody or antigen binding fragment thereof in any of the therapeutic methods or compositions disclosed herein.

Anti-human-PD-L1 antibodies (or VH and/or VL domains derived therefrom) suitable for use in the invention can be generated using methods well known in the art. Alternatively, art recognized anti-PD-L1 antibodies can be used. For example, human anti-PD-L1 antibodies disclosed in U.S. Pat. No. 7,943,743, the contents of which are hereby incorporated by reference, can be used. Such anti-PD-L1 antibodies include 3G10, 12A4 (also referred to as BMS-936559), 10A5, 5F8, 10H10, 1B12, 7H1, 11E6, 12B7, and 13G4. Other art recognized anti-PD-L1 antibodies which can be used include those described in, for example, U.S. Pat. Nos. 7,635,757 and 8,217,149, U.S. Publication No. 2009/0317368, and PCT Publication Nos. WO 2011/066389 and WO 2012/145493, each of which are herein incorporated by reference. Other examples of an anti-PD-L1 antibody include atezolizumab (TECENTRIQ; RG7446), or durvalumab (IMFINZI; MEDI4736). Antibodies or antigen binding fragments thereof that compete with any of these art-recognized antibodies or inhibitors for binding to PD-L1 also can be used.

Examples of anti-PD-L1 antibodies useful in the methods of the present disclosure include the antibodies disclosed in U.S. Pat. No. 9,580,507, which is herein incorporated by reference. Anti-PD-L1 human monoclonal antibodies disclosed in U.S. Pat. No. 9,580,507 have been demonstrated to exhibit one or more of the following characteristics: (a) bind to human PD-L1 with a KD of 1×10-7 M or less, as determined by surface plasmon resonance using a Biacore biosensor system; (b) increase T-cell proliferation in a Mixed Lymphocyte Reaction (MLR) assay; (c) increase interferon-γ production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present invention include monoclonal antibodies that bind specifically to human PD-L1 and exhibit at least one, in some embodiments, at least five, of the preceding characteristics.

In certain embodiments, the anti-PD-L1 antibody is BMS-936559 (formerly 12A4 or MDX-1105) (see, e.g., U.S. Pat. No. 7,943,743; WO 2013/173223). In other embodiments, the anti-PD-L1 antibody is MPDL3280A (also known as RG7446 and atezolizumab) (see, e.g., Herbst et al. 2013 J Clin Oncol 31(suppl):3000; U.S. Pat. No. 8,217,149), MEDI4736 (Khleif, 2013, In: Proceedings from the European Cancer Congress 2013; Sep. 27-Oct. 1, 2013; Amsterdam, The Netherlands. Abstract 802), or MSB0010718C (also called Avelumab; see US 2014/0341917). In certain embodiments, antibodies that cross-compete for binding to human PD-L1 with, or bind to the same epitope region of human PD-L1 as the above-references PD-L1 antibodies are mAbs. For administration to human subjects, these cross-competing antibodies can be chimeric antibodies, or can be humanized or human antibodies. Such chimeric, humanized or human mAbs can be prepared and isolated by methods well known in the art. In certain embodiments, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see, e.g., WO 2011/066389), avelumab (Pfizer; also known as BAVENCIO®, MSB-0010718C; see, e.g., WO 2013/079174), STI-1014 (Sorrento; see, e.g., WO2013/181634), CX-072 (Cytomx; see, e.g., WO2016/149201), KN035 (3D Med/Alphamab; see Zhang et al., Cell Discov. 7:3 (March 2017), LY3300054 (Eli Lilly Co.; see, e.g., WO 2017/034916), and CK-301 (Checkpoint Therapeutics; see Gorelik et al., AACR: Abstract 4606 (April 2016)). The above references are herein incorporated by reference.

In certain embodiments, the PD-L1 antibody is atezolizumab (TECENTRIQ®). Atezolizumab is a fully humanized IgG1 monoclonal anti-PD-L1 antibody.

In certain embodiments, the PD-L1 antibody is durvalumab (IMFINZI™). Durvalumab is a human IgG1 kappa monoclonal anti-PD-L1 antibody.

In certain embodiments, the PD-L1 antibody is avelumab (BAVENCIO®). Avelumab is a human IgG1 lambda monoclonal anti-PD-L1 antibody.

In other embodiments, the anti-PD-L1 monoclonal antibody is selected from the group consisting of 28-8, 28-1, 28-12, 29-8, 5H1, and any combination thereof.

Anti-PD-L1 antibodies usable in the disclosed methods also include isolated antibodies that bind specifically to human PD-L1 and cross-compete for binding to human PD-L1 with any anti-PD-L1 antibody disclosed herein, e.g., atezolizumab, durvalumab, and/or avelumab. In some embodiments, the anti-PD-L1 antibody binds the same epitope as any of the anti-PD-L1 antibodies described herein, e.g., atezolizumab, durvalumab, and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., atezolizumab and/or avelumab, by virtue of their binding to the same epitope region of PD-L1. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In certain embodiments, the antibodies that cross-compete for binding to human PD-L1 with, or bind to the same epitope region of human PD-L1 antibody as, atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies usable in the methods of the disclosed invention also include antigen-binding portions of the above antibodies. It has been amply demonstrated that the antigen-binding function of an antibody can be performed by fragments of a full-length antibody.

Anti-PD-L1 antibodies suitable for use in the disclosed methods or compositions are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain embodiments, the anti-PD-L1 antibody or antigen-binding portion thereof cross-competes with atezolizumab, durvalumab, and/or avelumab for binding to human PD-L1.

### Anti-CTLA-4 Antibodies

In certain embodiments, an embodiment encompasses use of an anti-CTLA-4 antibody. In one embodiment, the anti-CTLA-4 antibody binds to and inhibits CTLA-4. In some embodiments, the anti-CTLA-4 antibody is ipilimumab (YERVOY), tremelimumab (ticilimumab; CP-675,206), AGEN-1884, or ATOR-1015.

The invention will be now explained in detail by means of the following Examples and Figures.

### EXAMPLES

### Example 1: Antitumor Efficacy of Anti-CD36 Antibodies in Combination with PD1 Inhibition in C57B16/J Mice Bearing YUMM1.7 Cells-Derived Melanoma Tumors

250,000 YUMM1.7 cells are suspended in PBS and are injected subcutaneously in the flank of 8-12 week-old C57B16/J mice. When tumors reach a mean volume of 50-100 mm³, mice are randomized and the treatment is started.

The experimental groups are as shown in Table 1:

| Group | No. Mice | Treatment |
|---|---|---|
| 1 | 10 | anti-PD1 isotype control |
| | | (rat IgG2a, clone 2A3) |
| 2 | 10 | anti-mouse PD-1 |
| | | (clone RMP1-14) |
| 3 | 10 | anti-CD36 isotype control |
| 4 | 10 | anti-CD36 |
| 5 | 10 | anti-mouse PD-1 + anti CD-36 |

All antibodies are injected IP at the concentration of 10 mg/kg, 3 times/week. Mice are monitored three times per week for body weight and tumour volume and daily for behaviour and survival. When tumour reaches a maximum volume of 1.500 mm³, mice are euthanized and tissues collected. Primary tumours are weighted and measured again with a caliper. Lung and liver are embedded in paraffin for H&E staining and a blinded analysis for metastatic lesions.

### Example 2: Combination of Anti-CD36 Antibodies with Cisplatin

Studies on the combination of anti-CD36 antibody with cisplatin were performed in NSG mice (immuno-deficient) males and females (first experiment mice directly bought, follow up experiments in house breeding) All mice were inoculated with commercially available oral cancer cells Ab dosing was always done daily i.p.

Two types of oral cancer cell lines were inoculated:
Detroit: “medium” metastatic and very large primary tumor
FaDu: “very strong” metastatic and small primary tumor

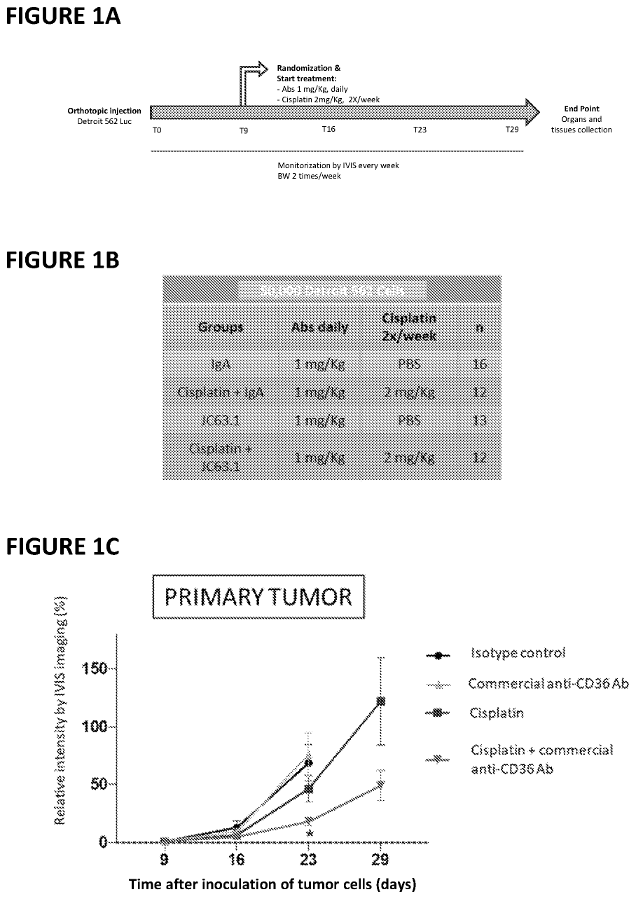

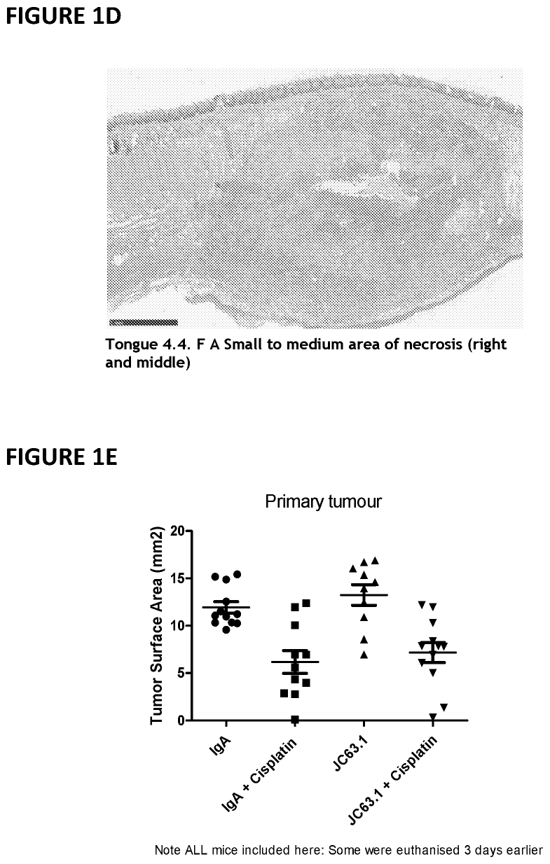

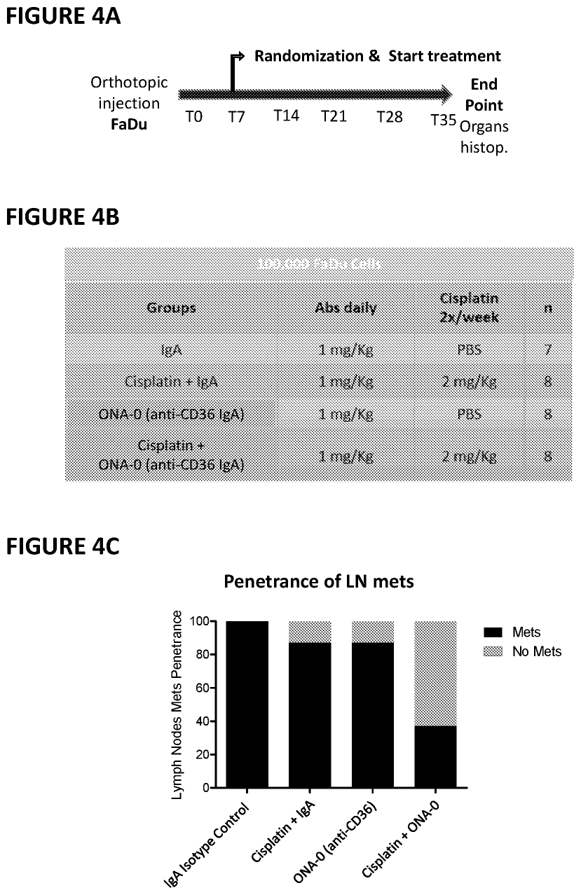

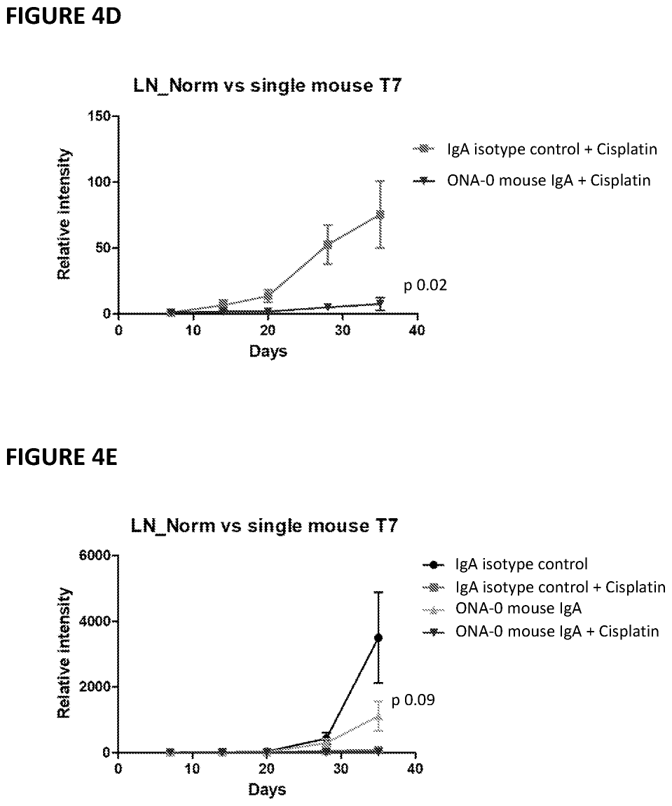

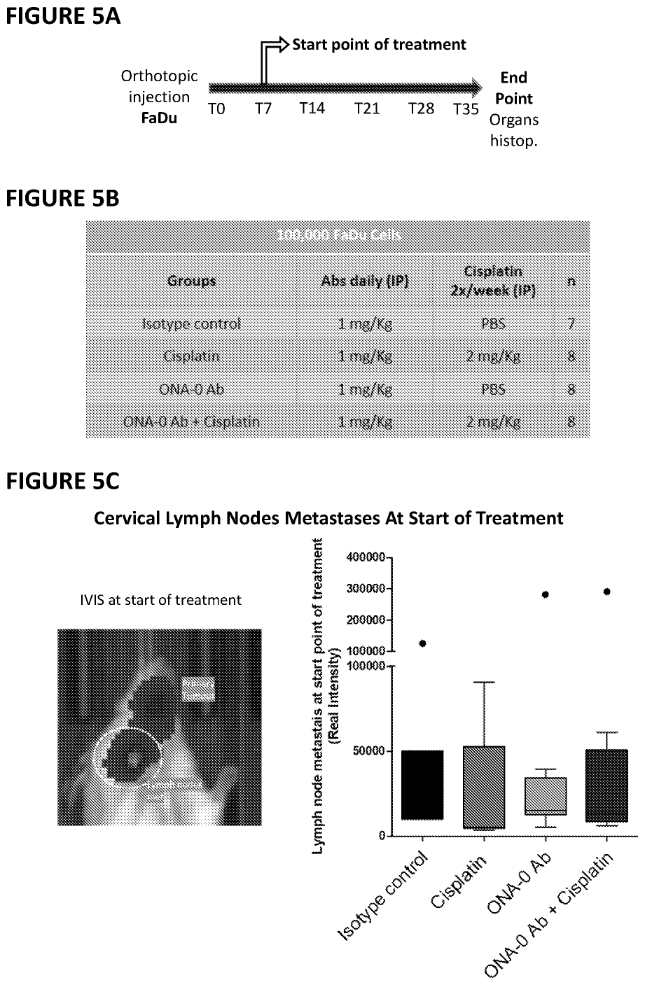

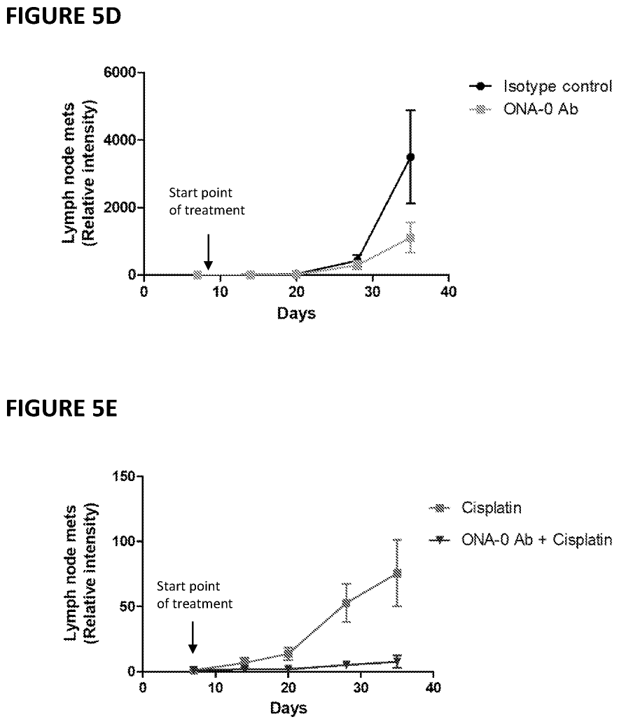

50,000 or 100,000 cancer cells were inoculated. The starting time of treatment was the amount of days after inoculation of the cancer cells Ab treatment starts. As can be seen in FIGS. 1A and 1B, the treatment groups for one study using Detroit cells were: Group 1: IgA, Group 2: cisplatin+IgA; Group 3: anti-CD36 antibody JC63.1; and group 4: anti-CD36 antibody JC63.1+cisplatin. As can be seen in FIGS. 4A and 4B, the treatment groups for a study using FaDu cells were: Group 1: IgA, Group 2: cisplatin+IgA; Group 3: anti-CD36 antibody ONA-0 (also known as ONA-0-v1; as described in U.S. Patent Application No. 63/117,529); and group 4: anti-CD36 antibody ONA-0+cisplatin. As can be seen in FIGS. 5A and 5B, the treatment groups for another study using FaDu cells were: Group 1: IgA, Group 2: cisplatin; Group 3: anti-CD36 antibody ONA-0; and group 4: anti-CD36 antibody ONA-0+cisplatin.

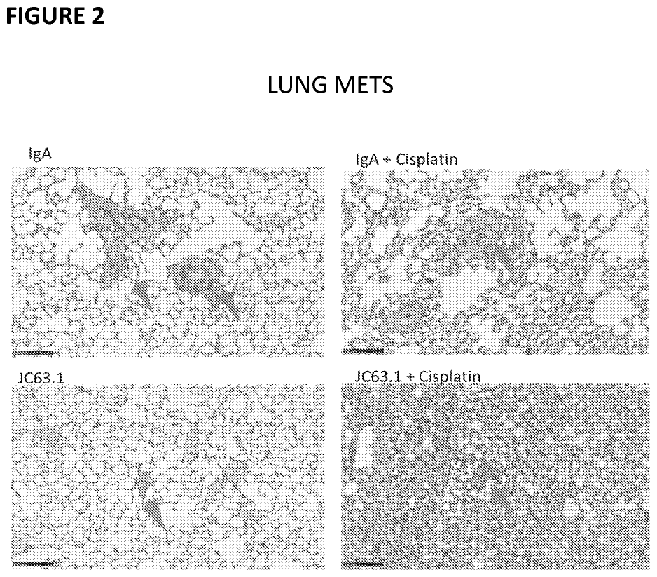

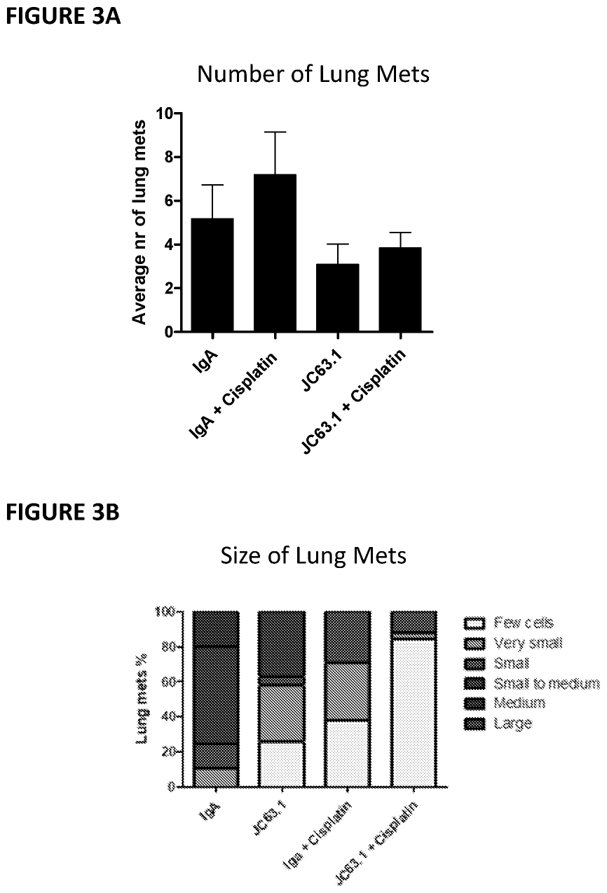

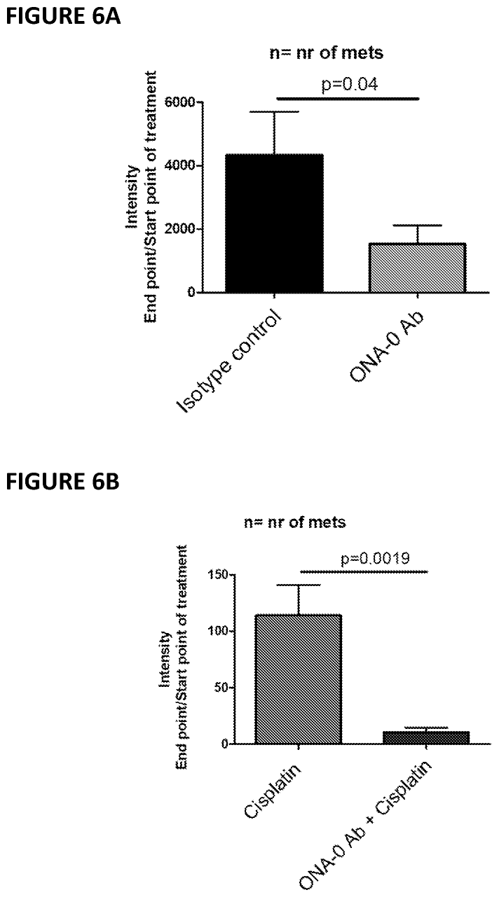

As can be seen in FIGS. 1C-1E, anti-CD36 Ab treatment has at least additive anti-tumor activity with cisplatin on the primary tumor in oral cancer. As can be seen in FIG. 2, combined anti-CD36 Ab and cisplatin treatment reduces the lung metastases in both size and number. As can be seen in FIGS. 3A-3B, anti-CD36 Ab treatment has a different method of action and complementary anti-tumor activity when combined with cisplatin in lung metastases from oral cancer: anti-CD36 Ab reduces the number and size of metastases, cisplatin reduces the size of metastases. As can be seen in FIGS. 4C-4E, anti-CD36 antibody demonstrates efficacy on lymph node metastases in mono and even more in combination therapy with cisplatin in the most aggressive FaDu cell line. FIGS. 5C-5E and FIGS. 6A-6B show lymph node metastasis in cisplatin treated mice, CD36 Ab treated mice, and cisplatin+CD36 Ab treated mice.

### Example 3: Treatment of Ovarian Cancer Using the ONA-0 Anti-CD36 Antibody in Combination with Cisplatin

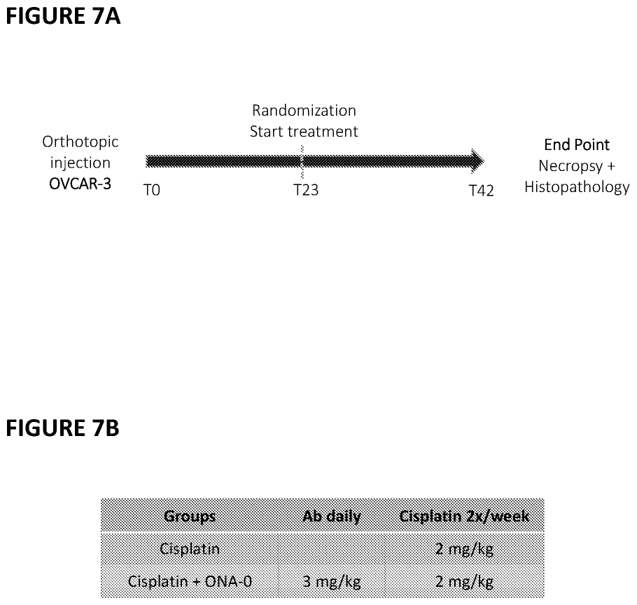

Studies of the effects of the combination of ONA-0 anti-CD36 antibody and cisplatin on ovarian cancer were performed in NSG mice (immuno-deficient). An experimental overview of these studies is provided in FIG. 7A. The studies included only female mice. All mice were inoculated with commercially available OVCAR-3 (ATCC) cancer cells. OVCAR-3 cells were derived from a human progressive adenocarcinoma of the ovary (i.e., from an ovarian cancer). Prior to inoculation, the OVCAR-3 cells were cultured in a humidified incubator at 37° C. with 5% CO₂, and were grown in RPMI-1640 supplemented with 5 μg ml⁻¹ penicillin/streptomycin, 0.01 mg/ml bovine insulin and 20% FBS (GIBCO).

For each mouse, a piece of an OVCAR-3 xenograft was implanted orthotopically. Treatment of the implanted mice began 23 days after implantation with the OVCAR-3 tumor pieces. Inoculated mice were divided into one of two treatment groups: cisplatin injection control (n=9) or cisplatin in combination with ONA-0 treatment (n=8). Antibody treatments were administered via intraperitoneal (i.p.) injection daily at a dose of 3 mg/kg and cisplatin treatments were administered via intraperitoneal (i.p.) injection twice per week at a dose of 2 mg/kg (FIG. 7B). Mice were sacrificed at the end of the treatment period. Upon sacrifice, organs and tissues were collected for performance of immunohistochemistry analysis.

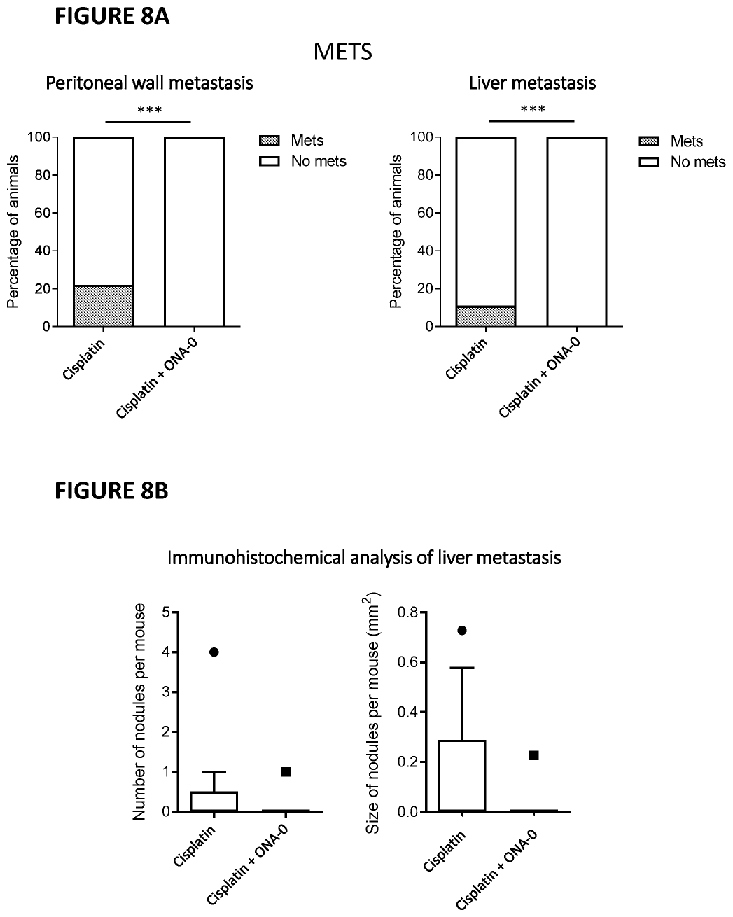

FIGS. 8A and 8B show the results of quantifying metastatic tumors in treated mice. FIG. 8A shows the results of macroscopic analysis of metastases in the peritoneal wall and liver, respectively. The presence of the metastases was evaluated by visual inspection. In the cisplatin-treated group, 22% of the animals had metastasis in the peritoneal wall while in cisplatin+ONA-0-treated animals, no metastasis were detected. In the liver, the percentage of mice with metastasis decreased from 11% in the cisplatin group to none in the treated group. In addition, treating with ONA-0 in addition to cisplatin decreased the number of liver metastasis and shifted the size of liver metastases such that fewer large metastases were found (FIG. 8B). Collectively, FIGS. 8A and 8B show that treatment with ONA-0 in combination with cisplatin is more is effective at reducing the formation and growth of metastases from ovarian cancer in comparison to cisplatin alone.

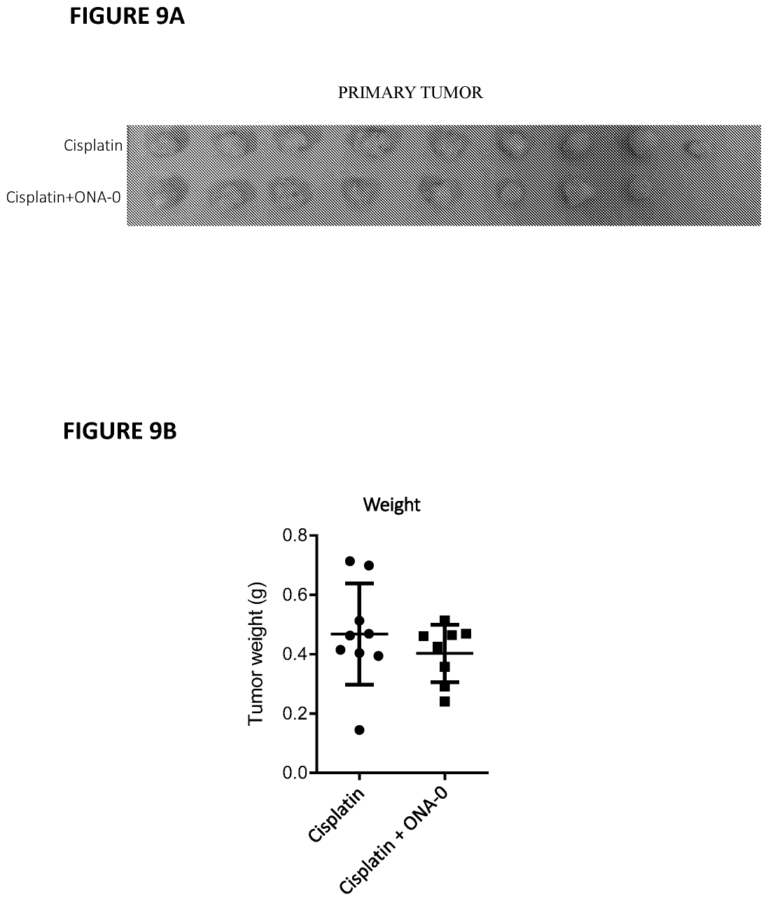

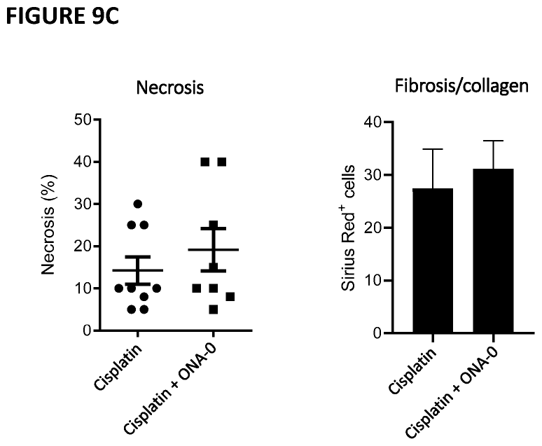

In addition to the effect on metastases, treatment with ONA-0 and cisplatin results in smaller primary tumors in the OVCAR-3 mouse model of ovarian cancer (FIG. 9A). The quantification of this effect in FIG. 9B shows that treatment with ONA-0 reduced tumor weight from an average of 0.468 grams to an average of 0.403 grams, a decrease of 14% percent. These data indicate that the combination inhibited tumor growth and/or promoted tumor cell destruction during the treatment period.

Histological analysis of the primary tumors in cisplatin-treated and cisplatin+ONA-0-treated mice was also performed. First, the tumors were analysed to determine percent necrosis by visual inspection and blinded quantification of a pathologist. The results of this analysis are shown in FIG. 9C, which shows that combination treatment increased necrosis from approximately 14.2% to approximately 19.3%. This increase indicates that combination treated tumors present higher amount of cell death. The primary tumors were also analysed to determine the percent of collagenous and fibrotic areas by Sirius red staining. The results of this analysis are shown in FIG. 9C, which shows that addition of ONA-0 to cisplatin increased the SR positive area from 27.45% to 31.15%. This increase indicates that treatment of cisplatin with ONA-0 increases fibrosis and, together with the increased necrosis, indicates that the combination-treated tumors and not only smaller, but also they are composed of fewer tumoral cells.

### Example 4: Treatment of Colon Cancer Using the 1G04 Anti-CD36 Antibody in Combination with Anti-PD-1 Antibody

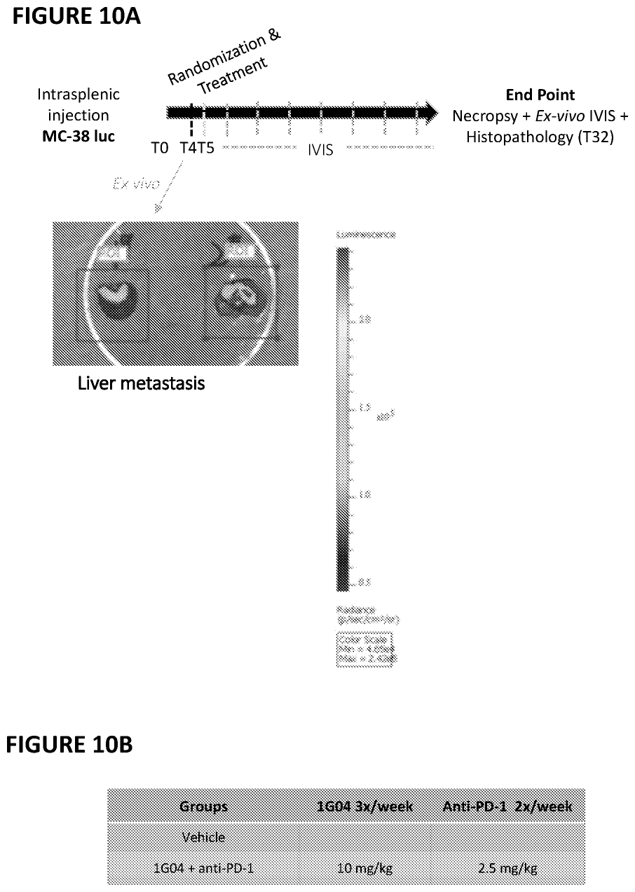

Studies of the effects of the combination of 1G04 anti-CD36 antibody (a chimeric version of the ONA-0 antibody, as described in U.S. Patent Application No. 63/117,529) and anti-PD-1 antibody (clone RMP1-14) on colon cancer were performed in C57BL/6 mice (immuno-competent). An experimental overview of these studies is provided in FIG. 10A. The studies included only female mice. All mice were inoculated with commercially available MC-38 cancer cells transduced with a viral vector expressing luciferase (MC-38-luc). MC-38 cells were derived from a murine colon adenocarcinoma (i.e., from a colon cancer). Prior to inoculation, the MC-38-luc cells were cultured in a humidified incubator at 37° C. with 5% CO₂, and were grown in DMEM supplemented with 0.5 μg ml⁻¹ puromycin and 10% FBS.

For each mouse, 1×10⁶ MC-38 cells were inoculated intrasplenically and the spleen was removed 5 minutes after injection. Four days after inoculation liver metastasis were confirmed ex vivo by luminescence and a representative image is shown in FIG. 10A. Treatment started five days after inoculation and inoculated mice were divided into two treatment groups: vehicle injection control (n=5) or anti-PD-1 in combination with 1G04 treatment (n=5). Treatments were administered via intraperitoneal (i.p.) injection, 1G04 was administered 3 times per week at a dose of 10 mg/kg and anti-PD-1 twice per week at a dose of 2.5 mg/kg (FIG. 10B). MC-38 cells are partially refractory to 2.5 mg/kg (and higher) doses of anti-PD-1 antibody. See, e.g., Fielder et al., Oncotarget 8:98371-98383 (2017); Chen et al., Cancer Immunology Research 3(2):149-160 (2015). During the course of treatment, mice were observed twice weekly using an in vivo imaging system (IVIS). Mice were sacrificed at the end of the treatment period. Upon sacrifice, organs and tissues were collected for performance of immunohistochemistry analysis.

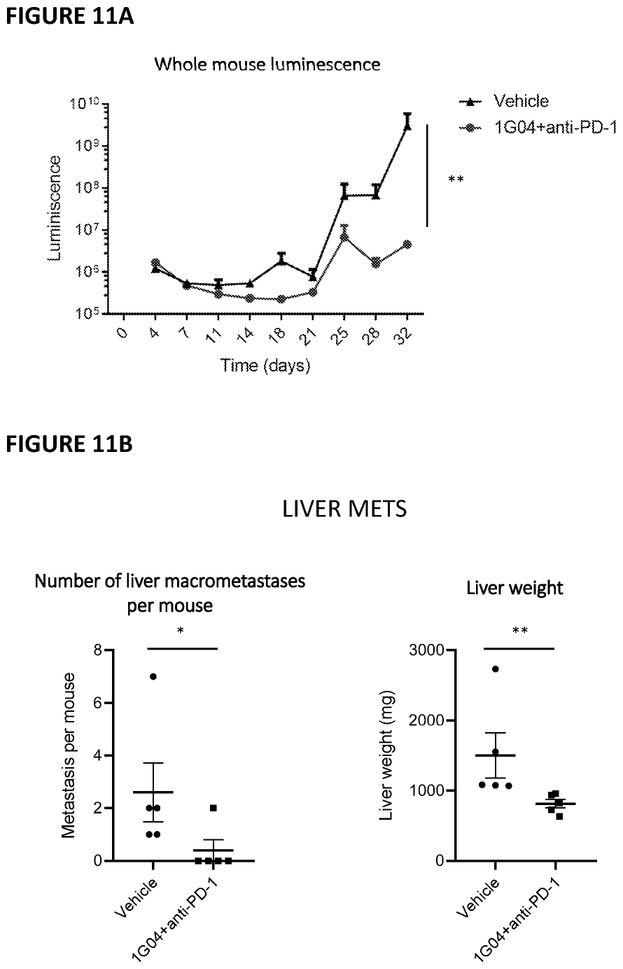

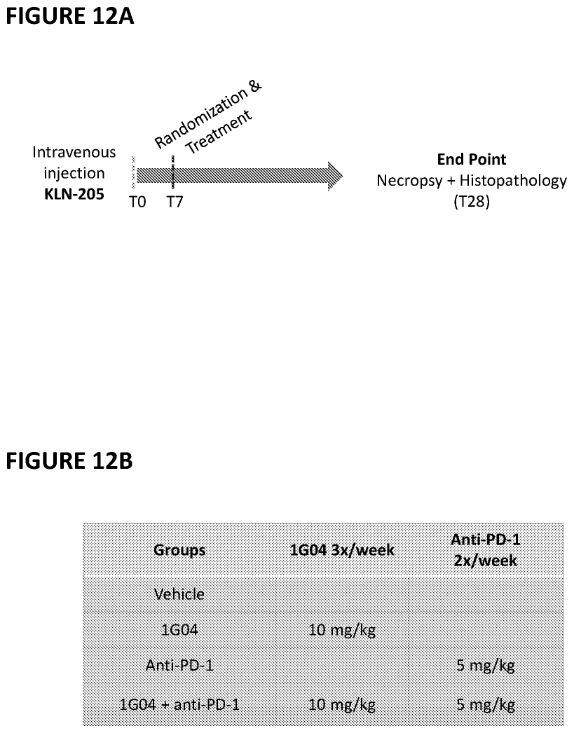

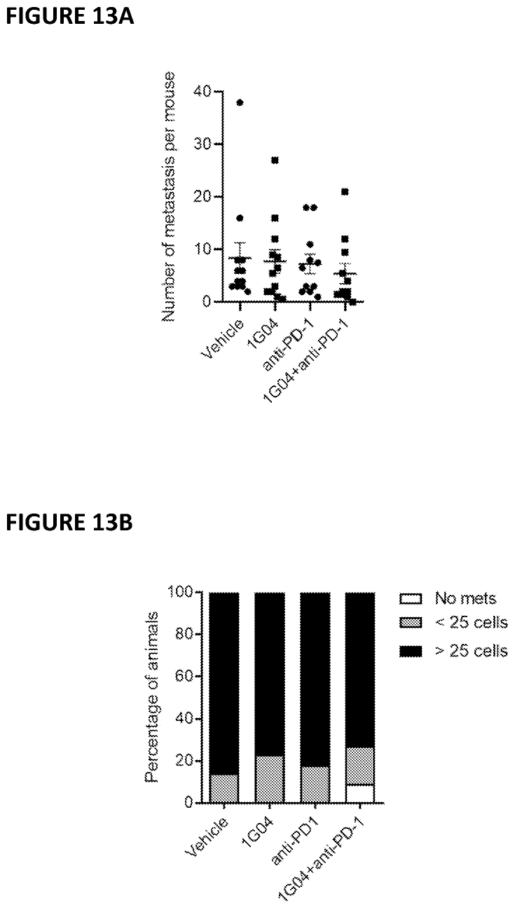

FIG. 11A shows the results of whole-animal bioluminescence imaging over time, which is a readout for the growth of luciferase-containing tumor cells in the mouse. The bioluminescence imaging showed that 1G04 in combination with anti-PD-1 decreased whole animal luminescence, and thus slowed the growth of the injected MC-38 tumor cells in vivo (**=p value of 0.0079).

FIG. 11B shows the results of macroscopic analysis of metastasis of the liver and liver weight. Treatment reduced the number of macrometastasis in the liver from 2.6 in the vehicle-treated group to 0.4 in the combination-treated mice (*=p value of 0.0397). Also, liver weight was reduced from 1.503 grams to 0.814 grams (46%) upon treatment with 1G04 and anti-PD-1 (**=p value of 0.0079). These results show that combination of the anti-CD36 antibody 1G04 with anti-PD-1 is efficient reducing the number of metastasis in the liver.

### Example 5: Treatment of Lung Cancer Using the 1G04 Anti-CD36 Antibody in Combination with Anti-PD-1 Antibody

Studies of the effects of the combination of 1G04 anti-CD36 and anti-PD-1 antibodies on lung cancer were performed in DBA/2 mice (immuno-competent). An experimental overview of these studies is provided in FIG. 12A. The studies included only female mice. All mice were inoculated with commercially available KLN-205 cancer cells that were derived from a murine lung squamous cell carcinoma (i.e., from a lung cancer). Prior to inoculation, the KLN-205 cells were cultured in a humidified incubator at 37° C. with 5% CO₂, and were grown in Eagle's Minimum Essential Medium supplemented with 10% FBS.

For each mouse, 2.5×10⁵ KLN-205 cells were inoculated intravenously in the tail vein. Treatment started seven days after inoculation and inoculated mice were divided into four treatment groups: vehicle injection control (n=12), 1G04 treatment (n=12), anti-PD-1 treatment (n=11) or the combination of 1G04 and anti-PD1 (n=11). Treatments were administered via intraperitoneal (i.p.) injection, 1G04 was administered 3 times per week at a dose of 10 mg/kg and anti-PD-1 twice per week at a dose of 5 mg/kg (FIG. 12B). KLN-205 tumors do not respond to such a 5 mg/kg dose or even to 10 mg/kg of anti-PD-1. See, e.g., Hai et al., Clinical Caner Research 26(13):3431-3442 (2020); Wu et al., JCI Insight 3(21):e124184 (2018). Mice were sacrificed at the end of the treatment period. Upon sacrifice, organs and tissues were collected for performance of immunohistochemistry analysis.

Blinded histological analysis of lung metastasis was performed by a pathologist. FIG. 13A shows that the total number of metastases decreased from a mean of 8.4 metastasis per mouse to 7.6 and 7.3 after 1G04 and anti-PD-1 treatment, respectively, while after treatment with 1G04 in combination with anti-PD-1 the mean number of metastasis was further decreased to 5.5 metastasis per mouse (35% reduction). FIG. 13B shows the results of analysing the size of metastases. In the vehicle-treated group, 86% of the animals had large metastasis (>25 cells per metastasis) and 14% had small to medium size metastasis (<25 cells per metastasis). In the 1G04 treated animals, 77% of mice had large metastasis and 23% of the animals had small-medium metastasis, while in the anti-PD-1 treated animals the respective percentages of affected mice were 82% and 18%. In the combination treated group, metastasis were reduced and 9% of the mice had no metastasis. Among the animals with metastasis, the number of large ones was reduced to 73% and medium-small ones to 18%. Treating with the single agents reduced the size of metastasis while the combination further reduced the size of the metastasis and also metastasis disappeared in a percentage of treated animals.

All references cited herein are incorporated by reference to the same extent as if each individual publication, database entry, patent application or patent was specifically indicated to be incorporated by reference.

1. A method of treating cancer in a subject comprising administering to the subject a therapeutically effective amount of:
a CD36 inhibitor; and
a second therapy.

2. The method of claim 1, wherein the cancer is selected from the group consisting of: oral squamous cell carcinoma, head and neck cancer, esophageal cancer, gastric cancer, ovarian cancer, cervical cancer, lung cancer, breast cancer, colon cancer, renal cancer, prostate cancer, sarcoma, melanoma, leukemia, and lymphoma.

3. The method of claim 1 or claim 2, wherein the cancer is metastatic cancer.

4. The method of claim 3, wherein the cancer comprises one or more metastatic tumors present in one or more of the liver, lung, spleen, kidney, cervical lymph nodes, or peritoneal wall.

5. The method of claim 1, wherein the cancer is a primary tumor.

6. The method of claim 1, wherein the subject is a human.

7. The method of claim 1, wherein the CD36 inhibitor is an antibody, a single chain antibody, or a scFv, Fab or F(ab′)2 fragment.

8. The method of claim 7, wherein the CD36 inhibitor is an antibody.

9. The method of claim 8, wherein the CD36 inhibitor is a humanized antibody.

10. The method of claim 8, wherein the CD36 inhibitor is a human antibody.

11. The method of claim 1, wherein the CD36 inhibitor is a shRNA or an iRNA, a siRNA, or an antisense RNA or DNA.

12. The method of claim 1, wherein the second therapy is an immunotherapy.

13. The method of claim 12, wherein the immunotherapy is a PD-1 inhibitor.

14. The method of claim 13, wherein the PD-1 inhibitor is an anti-PD-1 antibody.

15. The method of claim 14, wherein the anti-PD-1 antibody is pembrolizumab (KEYTRUDA; MK-3475), pidilizumab (CT-011), or nivolumab (OPDIVO; BMS-936558).

16. The method of claim 12, wherein the immunotherapy is a PD-L1 inhibitor.

17. The method of claim 16, wherein the PD-L1 inhibitor is an anti-PD-L1 antibody.

18. The method of claim 17, wherein the anti-PD-L1 antibody is atezolizumab (Tecentriq or RG7446), durvalumab (Imfinzi or MEDI4736), avelumab (Bavencio) or BMS-936559

19. The method of claim 12, wherein the immunotherapy is a CTLA-4 inhibitor.

20. The method of claim 19, wherein the CTLA-4 inhibitor is an anti-CTLA-4 antibody.

21. The method of claim 20, wherein the anti-CTLA-4 antibody is ipilimumab or an antigen-binding fragment thereof.

22. The method of claim 1, wherein the second therapy is one or more chemotherapeutic agents.

23. The method of claim 22, wherein the chemotherapeutic agent is cisplatin.

24. The method of any one of claims 1-4, wherein metastasis is reduced or inhibited in the subject.

25. The method of claim 24, wherein the number of metastases is reduced.

26. The method of claim 24, wherein the growth of one or more tumors is inhibited.

27. The method of claim 24, wherein the growth of one or more metastatic tumors is inhibited.

28. The method of any one of claims 24-27, wherein the treatment reduces the size of metastatic tumors, as measured by IVIS imaging or H&E staining.

29. The method of claim 1, wherein the treatment increases the amount of necrosis in one or more tumors.

30. The method of claim 1, wherein the treatment increases the amount of fibrosis in one or more tumors.

31. The method of claim 1, wherein the two therapies are administered sequentially.

32. The method of claim 1, wherein the two therapies are administered simultaneously.
